(19) United States
(12) Patent Application Publication
(10) Pub. No.: US 2008/0075973 A1
(43) Pub. Date: Mar. 27, 2008

(54) Red Electroluminescent Compounds And Organic Electroluminescent Device Using The Same

(75) Inventors: Bong-Ok Kim, Seoul (KR); Chi-Sik Kim, Jeonra-BukDo (KR); Hoon Han, Kyeongki-Do (KR); Seong-Min Kim, Kyeongki-Do (KR); Jung-Yeon Kim, Kyeongki-Do (KR); Kyu-Sung Cho, Kyeongki-Do (KR); So-Young Jung, Seoul (KR); Seung-Soo Yun, Seoul (KR); Hyuck-Joo Kwon, Kyeongki-Do (KR); Young-Jun Cho, Seoul (KR); Young-Kwon Kim, Seoul (KR); Sung-Min Kim, Seoul (KR)

Correspondence Address: THE WEBB LAW FIRM, P.C., 700 KOPPERS BUILDING, 436 SEVENTH AVENUE, PITTSBURGH, PA, 15219, US

(73) Assignee: Gracel Display inc., Seongdong-Gu (KR)

(21) Appl. No.: 11/628,679

(22) PCT Filed: Feb. 4, 2005
(86) PCT No.: PCT/KR05/00354

§ 371 Date: Sep. 5, 2007

(30) Foreign Application Priority Data
Jun. 7, 2004 (KR) 10 2004 0041434

(51) Int. Cl. B32B 9/04; C07D 471/06; C07D 471/10

(52) U.S. Cl. 428/690; 546/14; 546/18; 546/42; 546/58; 546/71; 546/94

(57) ABSTRACT

The present invention is related to organic electroluminescent compounds, methods of their preparation, and electroluminescent devices adopting them as electroluminescent materials.

### TECHNICAL FIELD

The present invention is related to organic electroluminescent compounds indicated in terms of the following Chemical Formula 1, methods of their manufacture, and electroluminescent devices adopting them as electroluminescent materials:

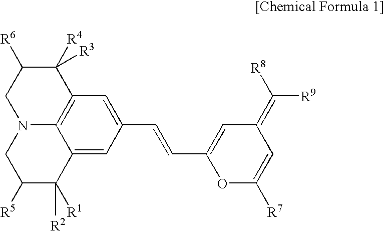

### BACKGROUND ART

The most important factor in the development of highly efficient and long-living organic EL devices is the development of high-performance electroluminescent materials. In reality, in view of the development of electroluminescent materials, red or blue electroluminescent materials have significantly low light-emitting characteristics compared to those of green electroluminescent materials. Three kinds of electroluminescent materials (i.e., red, green, and blue) are used in order to implement full-color display, which results in that the material having the lowest characteristics among three kinds of materials determines the performance of an entire panel. Therefore, the development of highly efficient and long-living blue or red electroluminescent materials is a critical subject for the improvement of characteristics of all organic EL devices.

The coloring purity and luminous efficiency of red electroluminescent materials known up to the present time have not been so much on a satisfactory level. In cases of most materials, the doping system has been used mainly since it has been difficult to construct high-performance electroluminescent devices using highly concentrated thin layers due to a concentration quenching effect among identical red electroluminescent molecules. That is, the farther the distance among molecules is, the more advantageous the light-emitting characteristics are. Also, it has not been easy to have highly efficient red light-emitting characteristics by lowering the sensitivity to colors in the pure red wavelength range of longer than 630 nm.

Accordingly, it may be possible to develop highly efficient and long-living red electroluminescent materials if only access among red electroluminescent molecules can be prevented and light-emitting wavelengths can be moved to longer wavelengths than those on the present level.

Among red electroluminescent materials, derivatives of DCM2 (4-(dicyanomethylene)-2-methyl-6-(julilodyl-9-enyl)-4H-pyran) have been known to be superior in view of their luminous efficiency and coloring purity. And the methods of using bulky substitution radicals for the minimization of access among molecules have been known in the studies related to the above derivatives of DCM2 in order to reduce the concentration quenching effect of red electroluminescent materials.

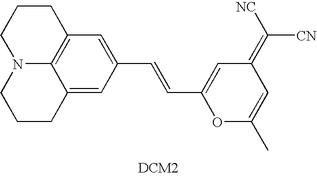

DCJTB (4-(dicyanomethylene)-2-tert-butyl-6-(1,1,7,7-tetramethyljulilodyl-9-enyl)-4H-pyran) showing the most superior efficiency among red electroluminescent materials reported up to the present time was published by C. H. Chen of Eastman-Kodak Company. This material was developed having DCJT (4-(dicyanomethylene)-2-methyl-6-(1,1,7,7-tetramethyljulilodyl-9-enyl)-4H-pyran) as the same frame with a concept of introducing a bulky substitution radical. In case of DCJTB, it was not only seen that the internal quenching effect was lowered rapidly due to a material in which the methyl radical of DCJT was transformed to a bulky tert-butyl radical, but also confirmed that DCJTB was improved remarkably in view of the wavelengths or luminous efficiency.

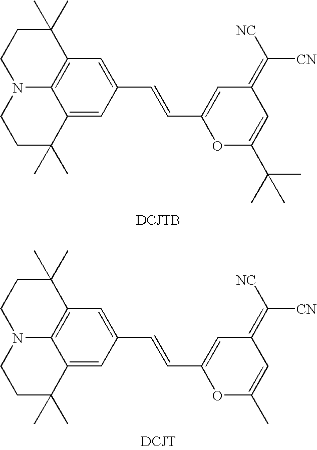

Also reported was a material called DCJTI (4-(dicyanomethylene)-2-isopropyl-6-(1,1,7,7-tetramethyljulilodyl-9-enyl)-4H-pyran) in the same group, in which the methyl radical in DCJT was transformed to an isopropyl radical.

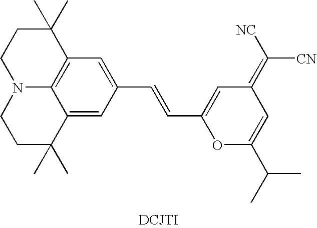

In the meantime, the inventors of the present invention have developed a high-performance red electroluminescent material having proper light-emitting characteristics by introducing a bulky substituent, such as adamantyl, 4-pentylbicyclo[2,2,2]octyl, etc., which is a fused ring, at position 2 of the conventional 4-(dicyanomethylene)-6-(1,1,7,7-tetramethyljulilodyl-9-enyl)-4H-pyran structure, and disclosed the invention in Korean Laid-Open Patent No. 2004-93679.

### DETAILED DESCRIPTION OF THE INVENTION

### Technical Problems

The inventors of the present invention have continued studies in order to develop electroluminescent materials having superior light-emitting characteristics compared to the conventional red electroluminescent materials. And they have realized that it has been possible to develop high-performance red electroluminescent materials by (i) preventing access among electroluminescent molecules, and (ii) grafting an idea, that could have moved light-emitting wavelengths of electroluminescent materials to long wavelengths, to designing of electroluminescent material molecules, and using the affects of polar energy that has been induced by the julilodyl radical, which has been an electron donor moiety, and the pyran part, which has been an electron acceptor moiety. This has enabled them to develop electroluminescent materials having more superior light-emitting characteristics than those of the conventional red electroluminescent materials by introducing substitution radicals of specific properties causing steric hindrance at a specific position of the julilodyl radical, which has been an electron donor moiety.

Accordingly, an object of the present invention is to provide with red electroluminescent compounds having a superior luminous efficiency even at a high concentration, and to provide with organic electroluminescent devices adopting the above electroluminescent compounds.

### Technical Methods of Resolution

The present invention is related to organic electroluminescent compounds, methods of manufacture thereof, and electroluminescent devices adopting them as electroluminescent materials.

The organic electroluminescent compounds according to the present invention have increased properties of the planar structure by having a fused ring, that can induce steric hindrance, introduced to the julilodyl radical; steric hindrance that can act advantageously in the access among molecules in solid thin layers; and significantly increased luminous efficiency through an efficient energy delivery mechanism. Generally, DCJTB, which has been a red fluorescent material, has been disadvantageous in that not only the luminous efficiency has been lowered due to trapping of the electric current, i.e., the carrier, by the electroluminescent dopant molecule during doping to the host, but also luminance has been reduced since the amount of charging flowing through the entire device has been reduced. Paying attention to the fact that such disadvantages could be removed by introducing functional radicals that could increase electrical conductivity to the dopant, the inventors of the present invention improved greatly the disadvantages of the conventional DCJTB through the improvement of electrical conductivity by introducing a silyl radical or an alkylsilyl radical.

Organic electroluminescent compounds according to the present invention are organic compounds shown in terms of the following Chemical Formula 1 concretely:

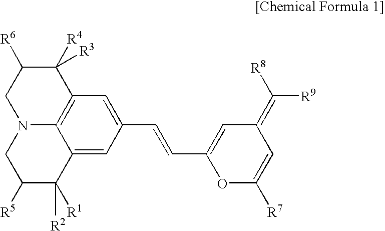

where R¹, R², R³, R⁴, R⁵, and R⁶ are independent from each other, and each of them may be hydrogen, a side-chained or straight-chained alkyl radical having a chain length of C₁ to C₁₀, cycloalkyl radical of C₅ to C₇, allyl radical, aralkyl radical, fused ring, or R¹¹R¹²R¹³Si—, where R¹ and R² or R³ and R⁴ are connected to C₅ to C₁₀ alkylene thus forming a spiro ring;

R¹ and R⁵ or R³ and R⁶ may form a fused ring as they are connected to C₃ to C₅ alkylene, and carbons of the alkylene of the fused ring connected to the above alkylene are substituted with R¹⁴R¹⁵Si< and may form a fused silacycloalkyl radical;

the alkyl radical, cycloalkyl radical, allyl radical, and aralkyl radical of the above R¹, R², R³, R⁴, R⁵, and R⁶ may be additionally substituted with more than one R¹¹R¹²R¹³Si—;

R⁷ is hydrogen or a side-chained or straight-chained alkyl radical having a chain length of C₁ to C₁₀, cycloalkyl radical of C₅ to C₇, allyl radical, aralkyl radical, or fused ring;

both of R⁸ and R⁹ is —CN or forms a 1,3-indandione ring as they are combined with

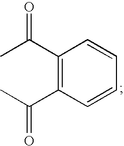

R¹¹, R¹², R¹³, R¹⁴ and R¹⁵ are the same as or different from each other, and each of them may be a side-chained or straight-chained alkyl radical having a chain length of C₁ to C₁₀, cycloalkyl radical of C₅ to C₇, allyl radical, or aralkyl radical, where R¹¹ and R¹² or R¹⁴ and R¹⁵ are connected to alkylene or alkenylene of C₄ to C₁₀ thus forming a spiro ring.

When more than one of R¹, R², R³, and R⁴ include the above substituent, the remaining substituents among R¹, R², R³, and R⁴ may be hydrogen, or side-chained or straight-chained alkyl radicals having a chain length of C₁ to C₁₀ that may be substituted or non-substituted, provided that a radical, selected from side-chained or straight-chained alkyl radicals of C₁ to C₁₀ where all of four R¹, R², R³, and R⁴ substitution radicals are composed of hydrogens or of carbons and hydrogens only, is excluded.

The compounds shown in terms of Chemical Formula 1 according to the present invention include the compounds shown in terms of the following Chemical Formula 2:

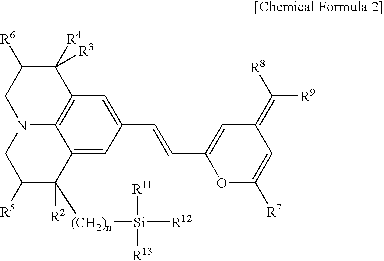

where n is an integer between 0 to 10;

R², R³, R⁴, R⁵, and R⁶ are independent from each other and each of them may be hydrogen, a side-chained or straight-chained alkyl radical having a chain length of C₁ to C₁₀, cycloalkyl radical of C₅ to C₇, allyl radical, aralkyl radical, fused ring, or R¹¹R¹²R¹³Si—, where R³ and R⁴ are connected to C₅ to C₁₀ alkylene thus forming a spiro ring;

R³ and R⁶ may form a fused ring as they are connected to C₃ to C₅ alkylene, and carbons of the alkylene of the fused ring connected to the above alkylene are substituted with R¹⁴R¹⁵Si< and may form a fused silacycloalkyl radical;

the above alkyl radical, cycloalkyl radical, allyl radical, arallyl radical, and fused ring may be additionally substituted with R¹¹R¹²R¹³Si—; and

R⁷, R⁸, and R⁹ are as shown in Chemical Formula 1.

Concrete examples of R², R³, R⁴, R⁵, and R⁶ of the compounds shown in terms of Chemical Formula 2 include mutually independent hydrogens, methyl, ethyl, n-propyl, i-propyl, n-butyl, i-butyl, t-butyl, n-pentyl, i-amyl, n-hexyl, n-heptyl, n-octyl, 2-ethylhexyl, n-nonyl, cycloalkyl radicals such as cyclopentyl, 2-methylcyclopentyl, 3-methylcyclohexyl, cycloheptyl, etc., phenyl, toluoyl, naphthyl, benzyl, 3-phenylpropyl, 2-phenylpropyl, adamantyl, 4-pentylbicyclo[2,2,2]octyl, norbornene, trimethylsilyl, triethylsilyl, tri(n-propyl)silyl, tri(i-propyl)silyl, tri(n-butyl)silyl, tri(i-butyl)silyl, tri(t-butyl)silyl, tri(n-pentyl)silyl, tri(i-amyl)silyl, t-butyldimethylsilyl, triphenylsilyl, tri(p-tolu)silyl, and dimethylcyclohexylsilyl;

where R¹¹ and R¹² may form methylsilacyclopentyl, methylsilacyclopentenyl, methylsilacyclohexyl, or ethylsilacyclohexyl radical connected to alkylene or alkenylene; and

the above alkyl radical, cycloalkyl radical, allyl radical, aralkyl radical, and fused ring may be additionally substituted with trimethylsilyl, triethylsilyl, tri(n-propyl)silyl, tri(i-propyl)silyl, tri(n-butyl)silyl, tri(i-butyl)silyl, tri(t-butyl)silyl, tri(n-pentyl)silyl, tri(i-amyl)silyl, t-butyldimethylsilyl, triphenylsilyl, tri(p-tolu)silyl, or dimethylcyclohexylsilyl.

In the meantime, the red electroluminescent compounds according to the present invention include the compounds forming fused rings shown in Chemical Formulas 3 and 4 as R¹ and R⁵ or R³ and R⁶ are connected to alkylene of C₃ to C₅ although they are independent from each other.

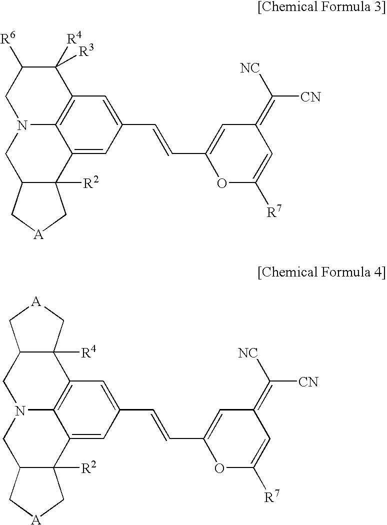

where substituents R², R³, R⁴, R⁶, and R⁷ are the same as those in Chemical Formula 1 or 2; ‘A’ may be mutually independent —CH₂—, —CH₂CH₂—, —CH₂CH₂CH₂—, etc.; and one of carbons in alkylene of the fused ring including ‘A’ may be substituted with R¹⁴R¹⁵Si<forming a fused silacycloalkyl radical. Concrete examples of thus formed silacycloalkyl radical include dimethylsilacyclopentane, ethylmethylsilacyclopentane, diethylsilacyclopentane, diphenylsilacyclopentane, dimethylsilacyclohexane, diethylsilacyclohexane, dipyhenylsilacyclohexane, etc., and R¹⁴ and R¹⁵ are connected to alkylene or alkenylene of C₄ to C₁₀ and include a spiro ring formed in the silacyclopentane, silacyclopentene, and silacyclohexane.

Silacycloalkanes of the above Chemical Formula 3 or 4 include organic electroluminescent compounds indicated in terms of the following Chemical Formula 5 or 6:

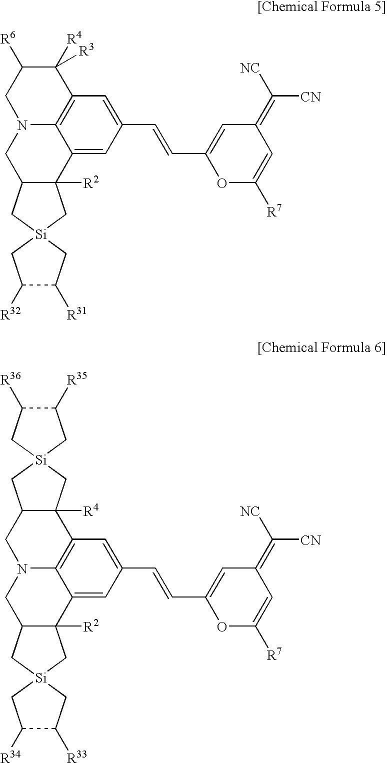

where “---” refers to a single bond or a double bond; R³¹, R³², R³³, R³⁴, R³⁵ and R³⁶ are independent from each other and are hydrogen, straight-chained or side-chained alkyl radical of C₁ to C₅; and R², R³, R⁴, R⁶, and R⁷ are the same as the substituents of Chemical Formula 1 or 2.

Also, the red electroluminescent compounds according to the present invention include the compounds forming spiro rings shown in Chemical Formulas 7 and 8 as R¹ and R² or R³ and R⁴ in the substituents of Chemical Formula 1 are independent from each other and connected to alkylene of C₃ to C₅.

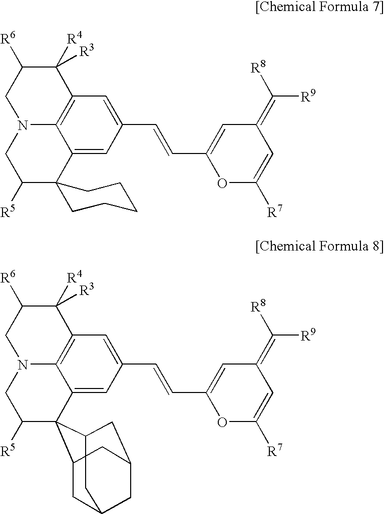

where R³, R⁴, R⁵, R⁶, R⁷, R⁸, and R⁹ are the same as those shown in Chemical Formula 2.

In the meantime, both of R⁸ and R⁹ may be —CN as shown in Chemical Formulas 3 to 6, or combined with 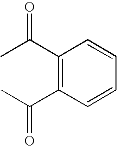
forming a 1,3-indandion ring, and further making the compounds shown in the following Chemical Formula 9:

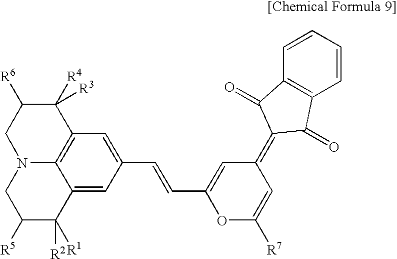

Concrete examples of R⁷ substituents of Chemical Formulas 1 through 9 include mutually independent hydrogen; side-chained or straight-chained alkyl radical having a chain length of C₁ to C₁₀ such as methyl, ethyl, n-propyl, i-propyl, n-butyl, i-butyl, t-butyl, n-pentyl, i-amyl, n-hexyl, n-heptyl, n-octyl, 2-ethylhexyl, and n-nonyl; cycloalkyl radical of C₅ to C₇ such as cyclopentyl, 2-methylcyclopentyl, 3-methylcyclohexyl, and cycloheptyl; allyl radical such as phenyl, toluoyl, and naphthyl; aralkyl radical such as benzyl, 3-phenylpropyl, and 2-phenylpropyl; and a fused ring such as adamantyl, 4-pentyl bicyclo[2,2,2]octyl, and norbornene. It is not desirable to have more than 20 carbon atoms in the above fused ring since it is likely that the electric conductivity may be degraded.

Tables 1 through 4 below show concrete examples of the range of red electroluminescent compounds according to the present invention:

TABLE 1

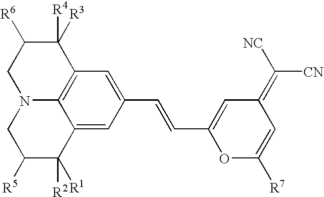

| Comp. | R₁ | R₂ | R₃ | R₄ | R₅ | R₆ | R₇ |
|---|---|---|---|---|---|---|---|
| 218 | 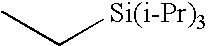 | H | —CH₃ | —CH₃ | H | H | 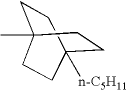 |
| 220 | 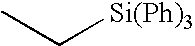 | H | —CH₃ | —CH₃ | H | H | 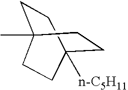 |
| 222 | 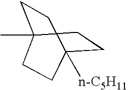 | —CH₃ | —CH₃ | —CH₃ | H | H | 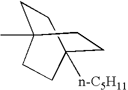 |
| 224 | 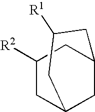 | | —CH₃ | —CH₃ | H | H | 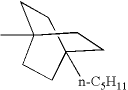 |
| 226 | 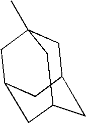 | —CH₃ | —CH₃ | —CH₃ | H | H | 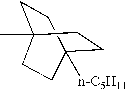 |
| 228 | 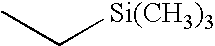 | H | —CH₃ | —CH₃ | H | H | 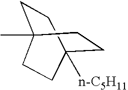 |
| 230 | 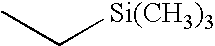 | —Si(CH₃)₃ | —CH₃ | —CH₃ | H | H | 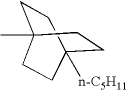 |
| 236 | 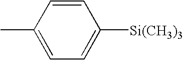 | —CH₃ | —CH₃ | —CH₃ | H | H | 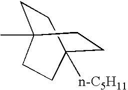 |
| 238 | 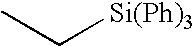 | —CH₃ | —CH₃ | —CH₃ | H | H | 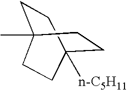 |
| 240 | 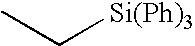 | H | 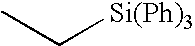 | H | H | H | 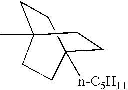 |
| 246 | 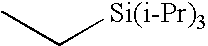 | —CH₃ | —CH₃ | —CH₃ | H | H | 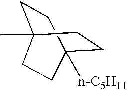 |
| 248 | 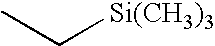 | —CH₃ | —CH₃ | —CH₃ | H | H | 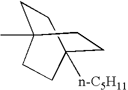 |
| 250 | 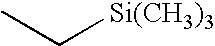 | H | 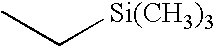 | H | H | H | 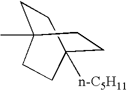 |
| 252 | 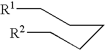 | | —CH₃ | —CH₃ | H | H | 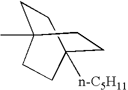 |
| 256 | 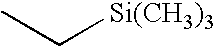 | 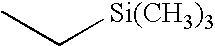 | —CH₃ | —CH₃ | H | H | 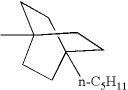 |
| 258 | —CH₃ | —CH₃ | —CH₃ | —CH₃ | 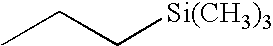 | H | 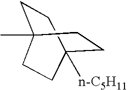 |
| 294 | 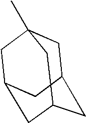 | H | —CH₃ | —CH₃ | H | H | 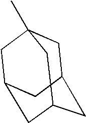 |
| 296 | 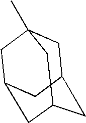 | H | —CH₃ | —CH₃ | H | H | t-Butyl |
| 302 | —Si(CH₃)₃ | 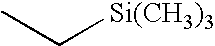 | —CH₃ | —CH₃ | H | H | 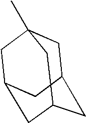 |
| 304 | —Si(CH₃)₃ | 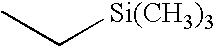 | —CH₃ | —CH₃ | H | H | 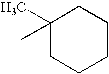 |
| 306 | —Si(CH₃)₃ | 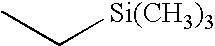 | —CH₃ | —CH₃ | H | H | 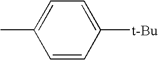 |
| 308 | —Si(CH₃)₃ | 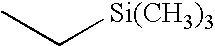 | —CH₃ | —CH₃ | H | H | 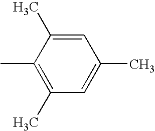 |
| 310 | —Si(CH₃)₃ | 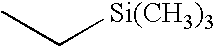 | —CH₃ | —CH₃ | H | H | t-Butyl |
| 218T | 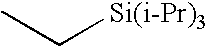 | H | —CH₃ | —CH₃ | H | H | t-Butyl |
| 220T | 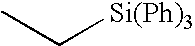 | H | —CH₃ | —CH₃ | H | H | t-Butyl |
| 222T | 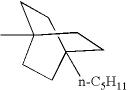 | —CH₃ | —CH₃ | —CH₃ | H | H | t-Butyl |
| 224T | 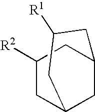 | | —CH₃ | —CH₃ | H | H | t-Butyl |
| 226T | 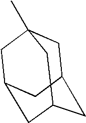 | —CH₃ | —CH₃ | —CH₃ | H | H | t-Butyl |
| 228T | 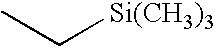 | H | —CH₃ | —CH₃ | H | H | t-Butyl |
| 230T | 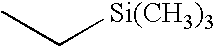 | —CH₃ | —CH₃ | —CH₃ | H | H | t-Butyl |
| 236T | 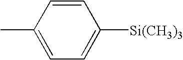 | —CH₃ | —CH₃ | —CH₃ | H | H | t-Butyl |
| 238T | 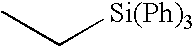 | —CH₃ | —CH₃ | —CH₃ | H | H | t-Butyl |
| 240T | 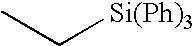 | H | 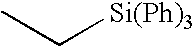 | H | H | H | t-Butyl |
| 246T | 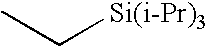 | —CH₃ | —CH₃ | —CH₃ | H | H | t-Butyl |
| 248T | 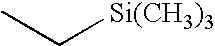 | —CH₃ | —CH₃ | —CH₃ | H | H | t-Butyl |
| 250T | 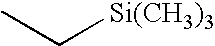 | H | 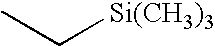 | H | H | H | t-Butyl |
| 252T | 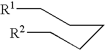 | | —CH₃ | —CH₃ | H | H | t-Butyl |
| 256T | 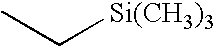 | 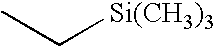 | —CH₃ | —CH₃ | H | H | t-Butyl |
| 258T | —CH₃ | —CH₃ | —CH₃ | —CH₃ | 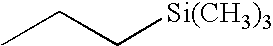 | H | t-Butyl |
| 294T | 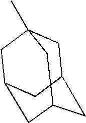 | H | —CH₃ | —CH₃ | H | H | t-Butyl |
| 218A | 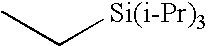 | H | —CH₃ | —CH₃ | H | H | 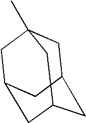 |
| 220A | 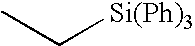 | H | —CH₃ | —CH₃ | H | H | 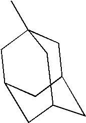 |
| 222A | 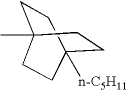 | —CH₃ | —CH₃ | —CH₃ | H | H | 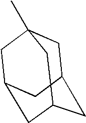 |
| 224A | 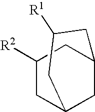 | | —CH₃ | —CH₃ | H | H | 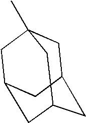 |
| 226A | 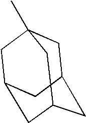 | —CH₃ | —CH₃ | —CH₃ | H | H | 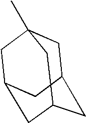 |
| 228A | 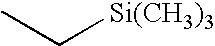 | H | —CH₃ | —CH₃ | H | H | 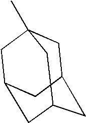 |
| 230A | 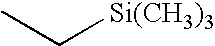 | —Si(CH₃)₃ | —CH₃ | —CH₃ | H | H | 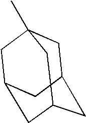 |
| 236A | 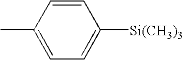 | —CH₃ | —CH₃ | —CH₃ | H | H | 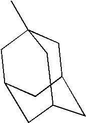 |
| 238A | 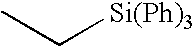 | —CH₃ | —CH₃ | —CH₃ | H | H | 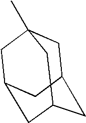 |
| 240A | 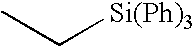 | H | 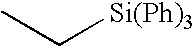 | H | H | H | 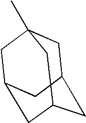 |
| 246A | 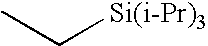 | —CH₃ | —CH₃ | —CH₃ | H | H | 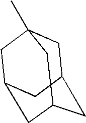 |
| 248A | 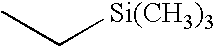 | —CH₃ | —CH₃ | —CH₃ | H | H | 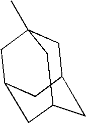 |
| 250A | 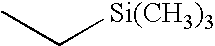 | H | 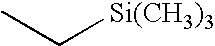 | H | H | H | 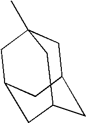 |
| 252A | 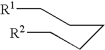 | | —CH₃ | —CH₃ | H | H | 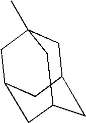 |
| 256A | 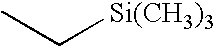 | 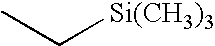 | —CH₃ | —CH₃ | H | H | 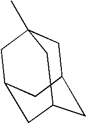 |
| 258A | —CH₃ | —CH₃ | —CH₃ | —CH₃ | 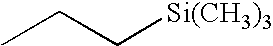 | H | 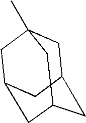 |

TABLE 2

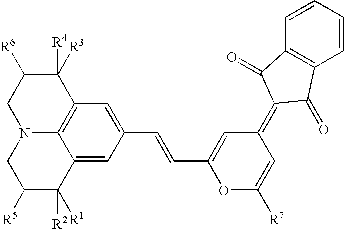

| Comp. | R₁ | R₂ | R₃ | R₄ | R₅ | R₆ | R₇ |
|---|---|---|---|---|---|---|---|
| 270 | —Si(CH₃)₃ | 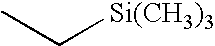 | —CH₃ | —CH₃ | H | H | 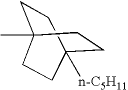 |
| 312 | —Si(CH₃)₃ | 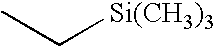 | —CH₃ | —CH₃ | H | H | 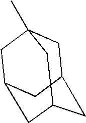 |
| 314 | —Si(CH₃)₃ | 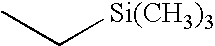 | —CH₃ | —CH₃ | H | H | 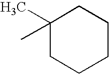 |
| 320 | —Si(CH₃)₃ | 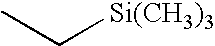 | —CH₃ | —CH₃ | H | H | t-Butyl |
| 219T | 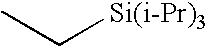 | H | —CH₃ | —CH₃ | H | H | t-Butyl |
| 239T | 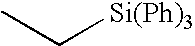 | —CH₃ | —CH₃ | —CH₃ | H | H | t-Butyl |
| 241T | 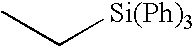 | H | 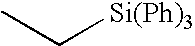 | H | H | H | t-Butyl |
| 247T | 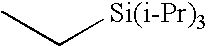 | —CH₃ | —CH₃ | —CH₃ | H | H | t-Butyl |
| 249T | 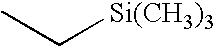 | —CH₃ | —CH₃ | —CH₃ | H | H | t-Butyl |
| 251T | 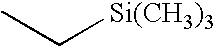 | H | 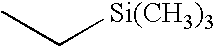 | H | H | H | t-Butyl |
| 219A | 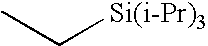 | H | —CH₃ | —CH₃ | H | H | 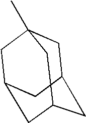 |
| 239A | 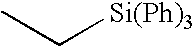 | —CH₃ | —CH₃ | —CH₃ | H | H | 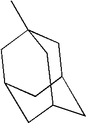 |
| 241A | 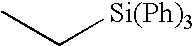 | H | 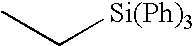 | H | H | H | 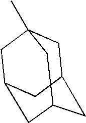 |
| 247A | 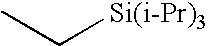 | —CH₃ | —CH₃ | —CH₃ | H | H | 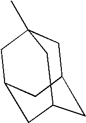 |
| 249A | 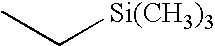 | —CH₃ | —CH₃ | —CH₃ | H | H | 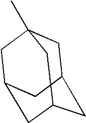 |
| 251A | 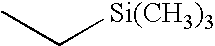 | H | 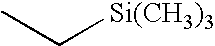 | H | H | H | 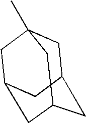 |

TABLE 3

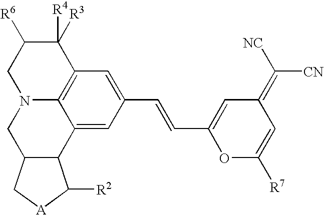

| Comp. | A | R₂ | R₃ | R₄ | R₆ | R₇ |
|---|---|---|---|---|---|---|
| 234 | —CH₂CH₂— | —CH₃ | —CH₃ | —CH₃ | H | 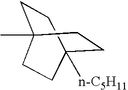 |
| 234T | —CH₂CH₂— | —CH₃ | —CH₃ | —CH₃ | H | t-Butyl |
| 234A | —CH₂CH₂— | —CH₃ | —CH₃ | —CH₃ | H | 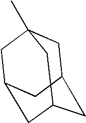 |
| 254 | —CH₂— | —CH₃ | —CH₃ | —CH₃ | H | 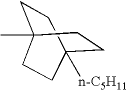 |
| 254T | —CH₂— | —CH₃ | —CH₃ | —CH₃ | H | t-Butyl |
| 254A | —CH₂— | —CH₃ | —CH₃ | —CH₃ | H | 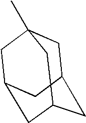 |
| 260 | 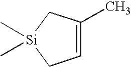 | —CH₃ | —CH₃ | —CH₃ | H | 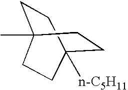 |
| 260T | 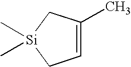 | —CH₃ | —CH₃ | —CH₃ | H | t-Butyl |
| 260A | 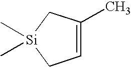 | —CH₃ | —CH₃ | —CH₃ | H | 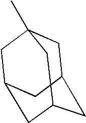 |

| | | | | | |
|---|---|---|---|---|---|
| 235 | —CH₂CH₂— | —CH₃ | —CH₃ | 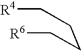 | 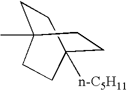 |
| 235T | —CH₂CH₂— | —CH₃ | —CH₃ | 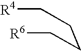 | t-Butyl |
| 235A | —CH₂CH₂— | —CH₃ | —CH₃ | 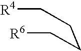 | 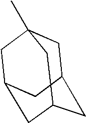 |
| 255 | —CH₂— | —CH₃ | —CH₃ | 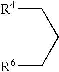 | 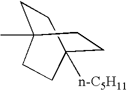 |
| 255T | —CH₂— | —CH₃ | —CH₃ | 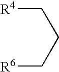 | t-Butyl |
| 255A | —CH₂— | —CH₃ | —CH₃ | 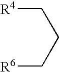 | 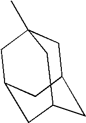 |
| 261 | 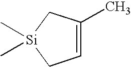 | —CH₃ | —CH₃ | 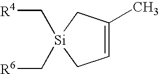 | 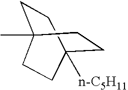 |
| 261T | 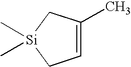 | —CH₃ | —CH₃ | 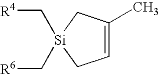 | t-Butyl |
| 261A | 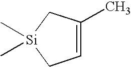 | —CH₃ | —CH₃ | 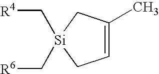 | 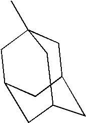 |

TABLE 4

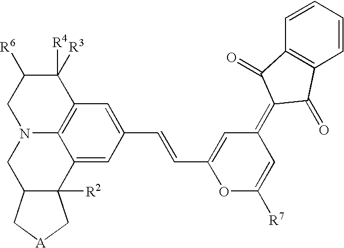

| Com- | | | | | | |
|---|---|---|---|---|---|---|
| pound | A | R₂ | R₃ | R₄ | R₆ | R₇ |
| 334 | —CH₂CH₂— | —CH₃ | —CH₃ | —CH₃ | H | 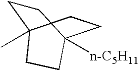 |
| 334T | —CH₂CH₂— | —CH₃ | —CH₃ | —CH₃ | H | t-Butyl |
| 334A | —CH₂CH₂— | —CH₃ | —CH₃ | —CH₃ | H | 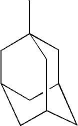 |
| 354 | —CH₂— | —CH₃ | —CH₃ | —CH₃ | H | 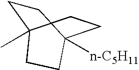 |
| 354T | —CH₂— | —CH₃ | —CH₃ | —CH₃ | H | t-Butyl |
| 354A | —CH₂— | —CH₃ | —CH₃ | —CH₃ | H | 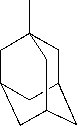 |
| 360 | 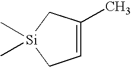 | —CH₃ | —CH₃ | —CH₃ | H | 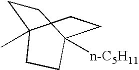 |
| 360T | 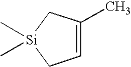 | —CH₃ | —CH₃ | —CH₃ | H | t-Butyl |
| 360A | 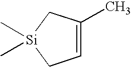 | —CH₃ | —CH₃ | —CH₃ | H | 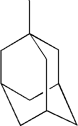 |

| | | | | | |
|---|---|---|---|---|---|
| 335 | —CH₂CH₂— | —CH₃ | —CH₃ | 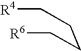 | 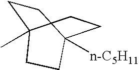 |
| 335T | —CH₂CH₂— | —CH₃ | —CH₃ | 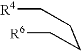 | t-Butyl |
| 335A | —CH₂CH₂— | —CH₃ | —CH₃ | 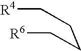 | 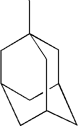 |
| 355 | —CH₂— | —CH₃ | —CH₃ | 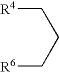 | 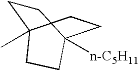 |
| 355T | —CH₂— | —CH₃ | —CH₃ | 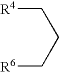 | t-Butyl |
| 335A | —CH₂— | —CH₃ | —CH₃ | 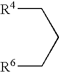 | 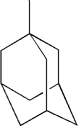 |
| 361 | 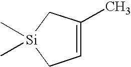 | —CH₃ | —CH₃ | 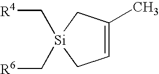 | 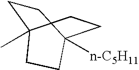 |
| 361T | 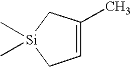 | —CH₃ | —CH₃ | 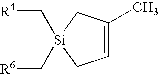 | t-Butyl |
| 361A | 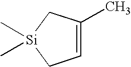 | —CH₃ | —CH₃ | 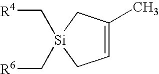 | 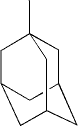 |

The method of manufacture of red electroluminescent compounds according to the present invention is illustrated below with reference to the following Chemical Equations 1 through 3. Chemical Equation 1 shows the step of reaction manufacturing julilodyl derivatives that are the electron donor moieties of the compounds according to the present invention.

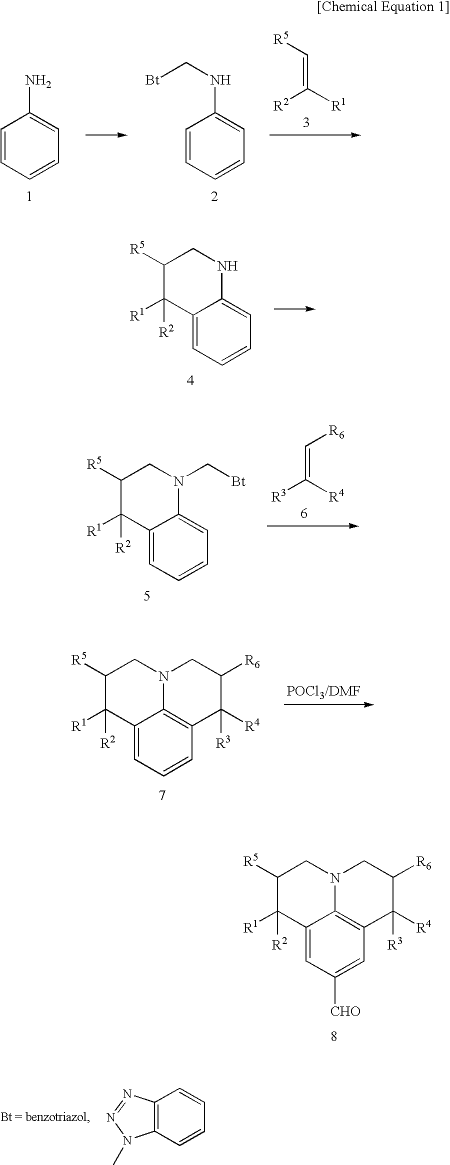

Aniline (1), which is a starting material, is dehydrated by using Dean-Stark reaction equipment, etc. along with 1H-benzotriazol-methanol or the mixed solution of benzotriazol and formaldehyde in order to make aniline (2) with benzotriazolyl methane substituted, after which a tetrahydroquinoline derivative (4) is made through ring formation according to Friedel-Crafts alkylation between the aniline derivative (2) and alkene derivative (3). During the ring formation, it is preferable to react them at a low temperature of about −78° C. under the catalyst of SnCl₄.

After making a tetrahydroquinoline derivative (5) with benzotriazolyl methane substituted through the substitution of benzotriazolyl methane again at the positions of remaining hydrogens of the tetrahydroquinoline derivative (4), a julilodyl derivative (7) is made through Friedel-Crafts reaction progressed previously, and a julilodyl aldehyde (8) derivative is made by reacting the above compound (7) under the condition of POCl₃/DMF.

If it is desired to manufacture a julilodyl derivative where R¹=R³, R²=R⁴, and R⁵=R⁶, it may be possible to manufacture at a time by reacting the alkene derivative (3) after making di(benzotriazolylmethyl)phenyl amine (9) from aniline as shown in the following Chemical Equation 12:

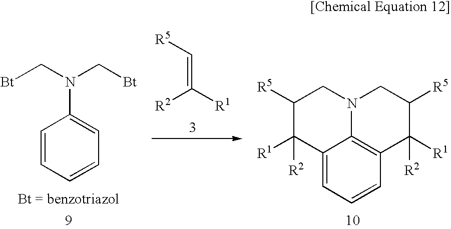

Next, the method of manufacture of a pyran derivative (16) or (18), which is an electron donor moiety, according to the present invention is illustrated with reference to

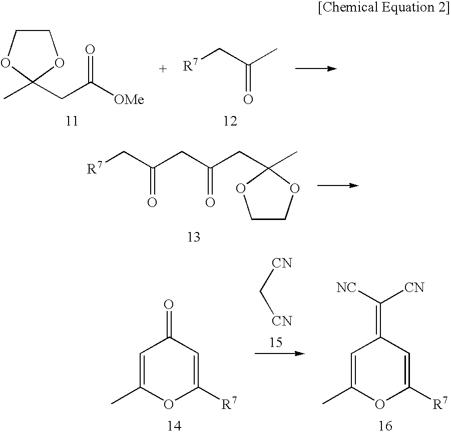

As shown in Chemical Equation 3, in manufacturing a pyran derivative, a triketone compound (13) with the ketone radical protected is manufactured through coupling of methyl acetoacetate (11) and a ketone derivative (12) with the ketone radical protected under the basic condition. Any base used generally is acceptable for the base to be used in the above step, but it is preferable to use a bulky base such as LDA, bis(trimethylsilyl)sodium amide (NaN(TMS)₂), etc. and the reaction is progressed at a proper temperature selected according to the properties of the base to be used. After making a pyran derivative (14) through deprotection and ring formation of the triketone compound (13) thus manufactured in an acidic solution, it is reacted with malononitrile (15) under the acidic or basic condition in order to manufacture the electron acceptor moiety of the compound of the present invention.

In the meantime, a pyran derivative (18) with indandion substituted may be manufactured besides pyran derivatives with the dicyano radical substituted by reacting 1,3-indandion with a pyran derivative (14).

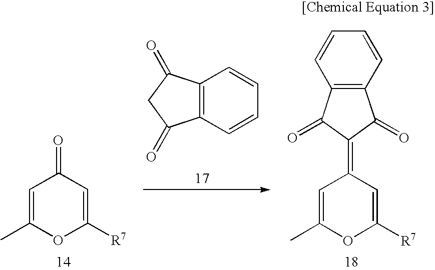

Red electroluminescent compounds according to the present invention are manufactured by reacting a julilodyl aldehyde derivative (8), which is an electron donor moiety manufactured in the above step, and a pyran derivative (16) or (18), which is an electron acceptor moiety, under the basic condition. Any general base is acceptable for the base to be used, but it is preferable to use a weak base such as piperidine, etc.

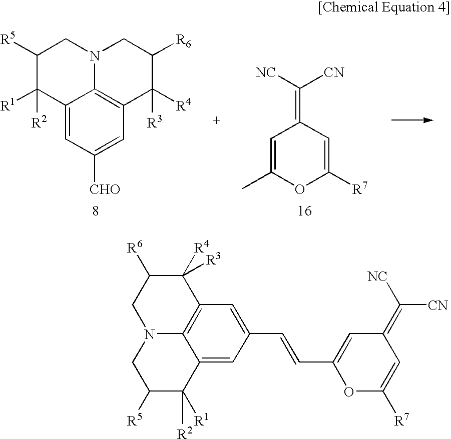

### BRIEF DESCRIPTION OF THE DRAWINGS

A more complete appreciation of this invention, and many of the attendant advantages thereof, will be readily apparent as the same becomes better understood by reference to the following detailed description when considered in conjunction with the accompanying drawings wherein:

FIG. 1 shows the structure of an organic EL device manufactured in Example 5;

FIG. 2 shows the electroluminescent spectrum of DCJTB shown in Chemical Formula b;

FIG. 3 shows the electroluminescent spectrum of Compound 234T synthesized in Example 3;

FIG. 4 shows the current density-voltage characteristic of Compound 234T described in Example 9;

FIG. 5 shows the luminance-voltage characteristic of Compound 234T described in Example 9;

FIG. 6 shows the luminous efficiency-luminance characteristic of Compound 234T described in Example 6; and

FIG. 7 shows the color coordinates-luminance characteristic of Compound 234T described in Example 7.

### EXAMPLES

Hereinafter, the methods of manufacture of red electroluminescent compounds according to the present invention are exemplified based on the Examples of the present invention, and the method of evaluation and results of evaluation of the characteristics of red electroluminescent compounds according to the present invention are presented.

### Example 1

### Synthesis of Compound 256

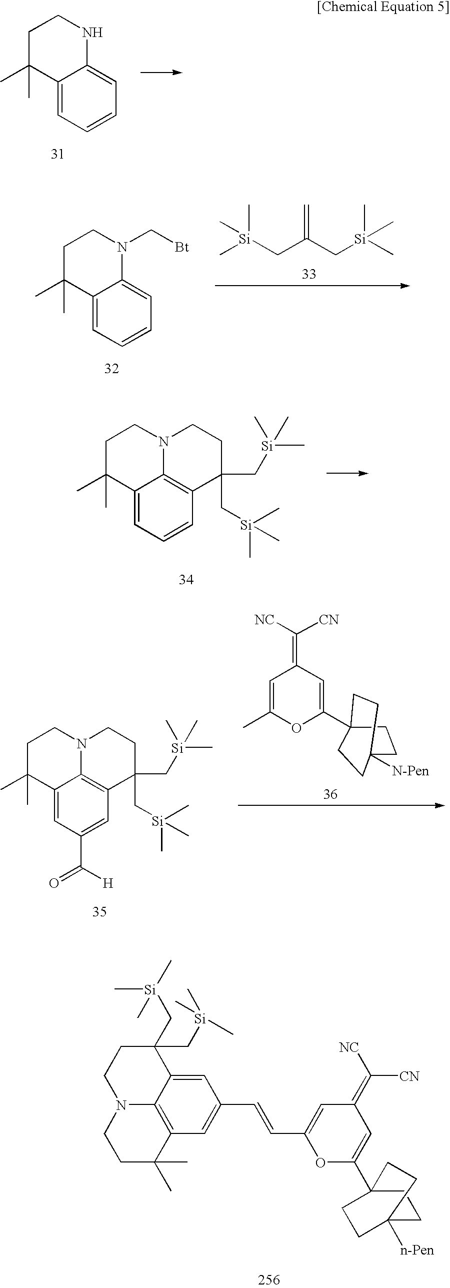

Subject Compound 256 is synthesized in the steps shown in Chemical Formula 5.

The 0.5 g portion (3.1 mmoles) of Compound 31, which is a tetrahydroquinolone derivative, 0.55 g (3.7 mmoles) of 1H-benzotriazole-1-methanol, and 1.5 g of molecular sieves (4 Å) are melted in 8 mL of THF, and heated at 50-60° C. until 1H-benzotriazole-1-methanol is melted completely. The heated material is stood still at a room temperature for 20 hours, after which molecular sieves are sifted and THF is blown off in order to obtain Compound 32.

To 10 ml of THF solution in which 2-methylene-1,3-propanediyl)-bis(trichlorosilane) (33) is dissolved, 34 mL of methyl lithium (1.6 M in diethylether) is added slowly under nitrogen. The mixture is stirred at a room temperature for 12 hours, and 10 mL of methanol is added slowly. The mixture is stirred for 10 minutes, and extracted with ether producing 0.52 g of Compound 33.

The 0.5 g portion (3.1 mmoles) of Compound 31, Compound 32, and 0.6 g (3.1 mmoles) of Compound 33 are melted in methylene chloride, and 3.1 mL of SnCl₄ (1.0 M in dichloromethane) is added under nitrogen at −78° C. The mixture is stirred at a room temperature for 12 hours. The reaction is terminated at 0° C. with the saturated NaOH aqueous solution, and the reaction mixture is extracted with methylene chloride producing 0.35 g of Compound 34.

Next, 0.27 mL (2.88 mmoles) of POCl₃ is put into 2 mL of DMF under nitrogen at 0° C., and this solution is stirred at a room temperature for 1 hour. 0.72 g (1.92 mmoles) of Compound 34 is melted in and added to 3 mL of DMF, and the reaction mixture is stirred at 40° C. for 12 hours. Then, the reaction is terminated with the saturated NaOH aqueous solution, and the reaction mixture is extracted with ethyl acetate producing 0.210 g of Compound 35.

The 0.358 g portion (0.89 mmole) of Compound 35 and 0.3 g (0.89 mmole) of Compound 36 are melted in 12 mL of ethanol, and 0.44 mL (4.45 mmoles) of piperidine is added to the mixture. Then, Dean-Stark trap filled with molecular sieves (4 Å) is installed, and the mixture is heated under nitrogen at 120° C. for 7 hours. After 12 hours, the reaction material is cooled to 0° C. and precipitates formed as the product of reaction are filtered, and recrystallized with methylene chloride and n-hexane thus producing 0.42 g (synthetic yield of 67%) of Subject Compound 256.

### Example 2

### Synthesis of Compound 248

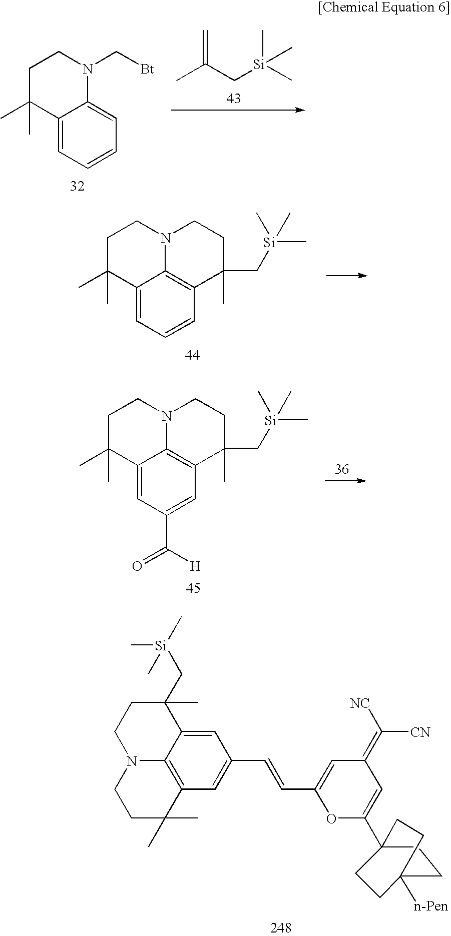

Subject Compound 248 is synthesized in the steps shown in Chemical Equation 6.

The 0.5 g portion (3.1 mmoles) of Compound 42 and 0.54 mL (3.1 mmoles) of methallyltrimethylsilane (43) are dissolved in methylene chloride, and 3.1 mL of SnCl₄ (1.0 M in dichloromethane) is added slowly to the mixed solution at −78° C. And 0.37 g of Compound 44 is obtained in the same method as that of synthesis of Compound 34 in Example 1. Then, 0.29 g of Compound 45 is obtained in the same method as that of synthesis of Compound 35 in Example 1 by using 0.37 g (1.22 mmoles) of Compound 44 synthesized in the above.

The 0.29 g portion (0.89 mmole) of Compound 45, 0.3 g (0.89 mmole) of Compound 36, and 0.44 mL (4.45 mmoles) of piperidine are dissolved in 12 mL of ethanol, and precipitates are obtained by reacting the mixed solution in the same method as that of synthesis of Compound 256 in Example 1. Then, these precipitates are recrystallized in methylene chloride and n-hexane, and 0.410 g (synthetic yield of 71%) of Compound 248, which is the subject compound, is obtained.

### Example 3

### Synthesis of Compound 260

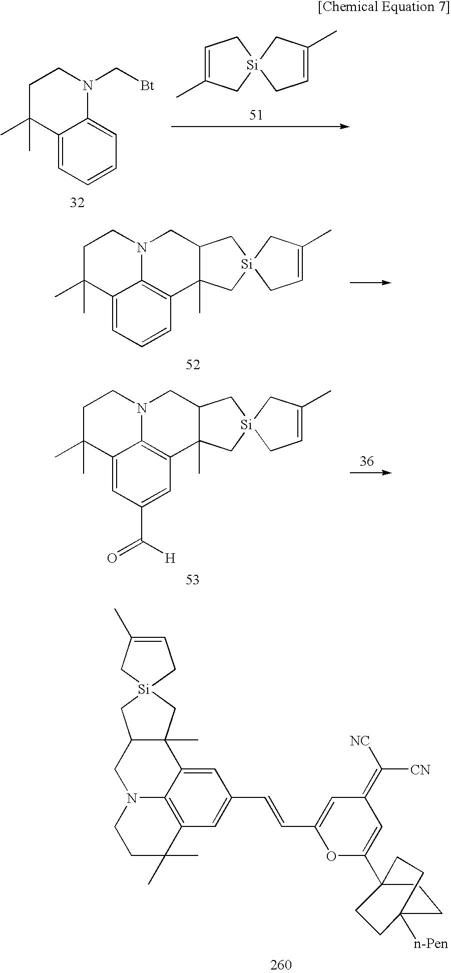

Subject Compound 260 is synthesized in the steps shown in Chemical Equation 7.

The 0.70 g portion of Compound 52 is obtained by using 0.50 g (3.1 mmoles) of Compound 31 and 0.54 mL (3.1 mmoles) of 2,7-dimethyl-5-silaspiro[4,4]-nona-2,7-diene (51) in the same method as that of synthesis of Compound 33 in Example 1. Then, 0.65 g of Compound 53 is obtained by using 0.70 g (2.07 mmoles) of Compound 52 thus obtained in the same method as that of synthesis of Compound 35.

Precipitates are obtained as the reaction product by using the mixed solution of 0.31 g (0.85 mmole) of Compound 53, 0.28 g (0.85 mmole) of Compound 36, and 0.42 mL (4.25 mmoles) of piperidine in 10 mL of ethanol in the same method as that of synthesis of Compound 256 in Example 1. These precipitates are recrystallized by using ethyl acetate, and 0.31 g (synthetic yield of 53%) of Compound 260, which is the subject compound, is obtained.

### Example 4

### Synthesis of Compound 258

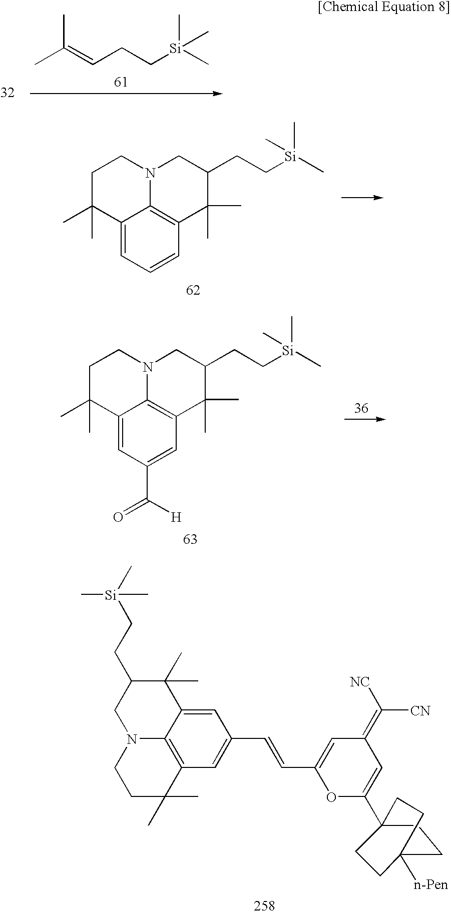

Subject Compound 258 is synthesized in the steps shown in Chemical Equation 8.

The 3.4 mL portion of (trimethylsilylmethyl)magnesium chloride (1.0 M in diethylether, 3.4 mmoles) is mixed with 10 mL of THF, and 4-bromo-2-methyl-2-butene dissolved in 5 mL of THF is added slowly to the solution. 0.49 g of Compound 61 is obtained by stirring the mixed solution at a room temperature for 12 hours, finishing the reaction with the NH₄Cl aqueous solution, and extracting the reaction product with ether. 0.40 g of Compound 62 is obtained by using 0.51 g (3.16 mmoles) of Compound 32 and 0.49 g (3.16 mmoles) of Compound 61 in the same method as that of synthesis of Compound 34 in Example 1. Also, 0.26 g of Compound 63 is obtained by using 0.40 g (1.2 mmoles) of this Compound 62 in the same method as that of synthesis of Compound 35 in Example 1.

Precipitates, which are the reaction product, are obtained by using the mixed solution of 0.26 g (0.72 mmole) of Compound 63, 0.24 g (0.72 mmoles) of Compound 36, and 0.36 mL (3.63 mmoles) of piperidine in 10 mL of ethanol in the same method as that of synthesis of Compound 256 in Example 1. Thus obtained precipitates are recrystallized with n-hexane and ethanol, and 0.25 g (synthetic yield of 51%) of Subject Compound 258 is obtained.

### Example 5

### Synthesis of Compound 250

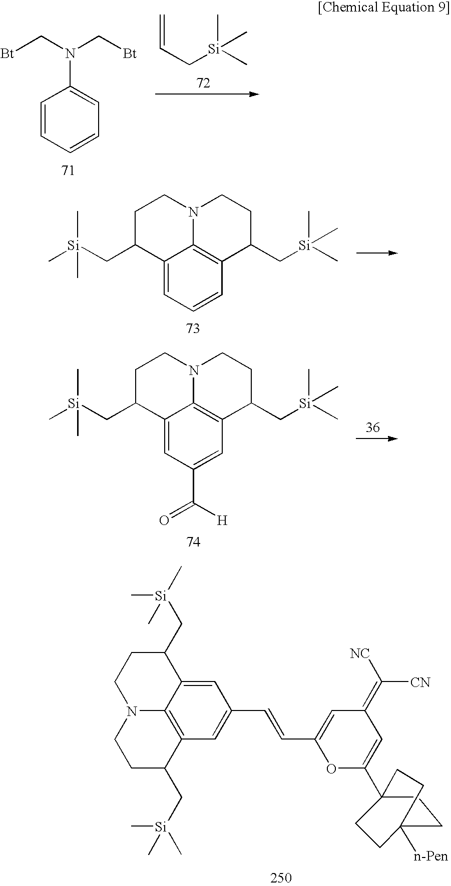

Subject Compound 250 is prepared in the steps shown in Chemical Equation 9.

The 0.58 mL portion (6.4 mmoles) of aniline, 2.3 g (19.3 mmoles) of benzotriazole, and 1.9 mL (37% aqueous solution, 25.7 mmoles) of formaldehyde are dissolved in 20 mL of toluene, and the mixed solution is refluxed for 12 hours by using Dean-Stark trap. It is then cooled to a room temperature, 10 mL of toluene is added, and the mixture is maintained at 0° C. for 24 hours. Compound 71 is obtained by filtering thus produced precipitates and removing toluene from the residual solution.

Compound 71 thus manufactured and 2.1 mL (12.9 mmoles) of allyltrimethylsilane (72) are dissolved in 20 mL of methylene chloride, and 12.8 mL (1.0 M in dichloromethane, 12.9 mmoles) of SnCl₄ is added slowly under nitrogen at −78° C. After obtaining 0.60 g of Compound 73 in the same method as that of synthesis of Compound 12, 0.28 g of Compound 74, which is the subject compound, is obtained by using 0.60 g (1.7 mmoles) of Compound 73 in the same method as that of synthesis of Compound 35 in Example 1.

The 0.30 g portion (0.78 mmole) of Compound 74, 0.27 g (0.78 mmole) of Compound 36, and 0.39 mL (3.94 mmoles) of piperidine are dissolved in 10 mL of ethanol. Then, 0.43 g (synthetic yield of 80%) of Compound 250, which is the subject compound, is obtained by refining precipitates, that are the reaction product obtained in the same method as that of synthesis of Compound 256, by means of column chromatography and recrystallization (methylene chloride, n-hexane).

### Example 6

### Synthesis of Compound 234T

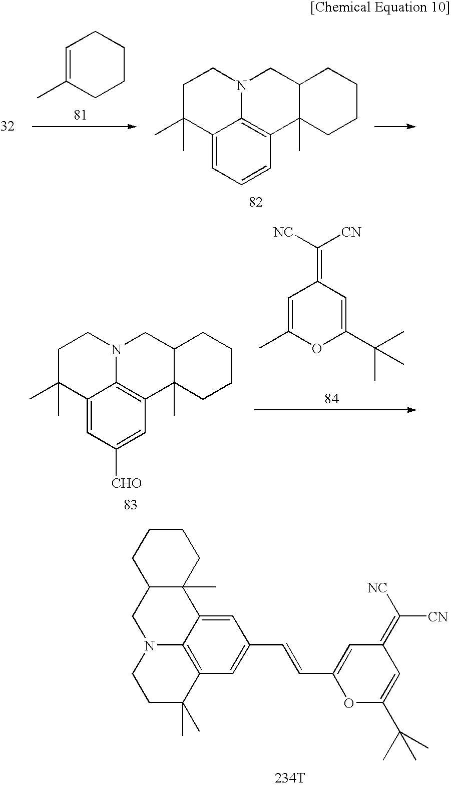

Subject Compound 234T is prepared in the steps shown in Chemical Equation 10.

The 0.49 g portion of Compound 82 is obtained by using 0.48 g (3.0 mmoles) of Compound 32 and 0.36 mL (3.0 mmoles) of 1-methyl-1-cyclohexene (81) in the same method as that of synthesis of Compound 34 in Example 1. 0.39 g of Compound 83 is obtained by using 0.49 g (1.8 mmoles) of thus obtained Compound 82 in the same method as that of synthesis of Compound 35 in Example 1.

Precipitates are obtained as the reaction product by dissolving 0.39 g (1.3 mmoles) of Compound 83, 0.28 g (1.3 mmoles) of Compound 84, and 0.6 mL (6.5 mmoles) of piperidine in 10 mL of ethanol and reacting them in the same method as that of synthesis of Compound 256 in Example 1. Then, 0.36 g (synthetic yield of 58%) of Compound 234T, which is the subject compound, is obtained through recrystallization of thus obtained precipitates with n-hexane and methylene chloride.

The data on m.p., ¹H-NMR, and mass spectrum of a part of compounds synthesized according to the methods of synthesized according to the present invention are presented in Table 5 below:

TABLE 5

| Compound | | | Mass |
|---|---|---|---|
| No. | mp (° C.) | ¹H NMR Analysis (δ) | Spectrum |
| 218 | 148 | | |
| 220 | 178 | 7.53-7.51(m, 6H), 7.47-7.44(m, 3H), 7.39-7.36(m, 6H), | 820.46 |
| | | 7.15(d, 1H), 7.08(d, 1H), 6.67(d, 1H), 6.52(d, 1H), | |
| | | 6.42(d, 1H), 6.07(d, 1H), 3.46-3.44(m, 1H), | |
| | | 3.34-3.20(m, 3H), 3.12-3.09(m. 1H), 1.89-1.83(m, 6H), | |
| | | 1.75-1.65(m, 4H), 1.57-1.54(m, 6H), 1.36(s, 3H), | |
| | | 1.29(s, 3H), 1.21(s, 2H), 1.36-1.20(m, 8H), 0.92(t, 3H) | |
| 222 | 200 | | |
| 224 | 288 | | |
| 226 | 268 | | |
| 228 | 238 | 7.25(d, 1H), 7.18(d, 1H), 7.06(s, 1H), 6.56(d, 1H), | 633.41 |
| | | 6.39(d, 1H), 3.29(m, 2H), 3.23(m, 1H), | |
| | | 1.87-1.84(m, 6H), 1.75(m, 4H), 1.52(m, 6H), | |
| | | 1.33(s, 3H), 1.30(s, 3H), 1.23-1.15(m, 8H), 0.97(m, 1H), | |
| | | 0.90(t, 3H), 0.91-0.87(m, 1H), 0.09(s, 9H) | |
| 230 | 206 | | |
| 234 | 266 | | |
| 236 | 198 | 7.43(d, 2H), 7.34(d, 1H), 7.22(d, 1H), 7.08(d, 2H), | 709.45 |
| | | 7.07(s, 1H), 6.52(d, 1H), 6.39(s, 1H), 6.37(d, 1H), | |
| | | 3.21(m, 2H), 3.09(m, 1H), 2.17(m, 2H), | |
| | | 1.80-1.78(m, 6H), 1.75(s, 3H), 1.51-1.48(m, 6H), | |
| | | 1.39(s, 3H), 1.34(s, 3H), 1.30-1.15(m, 8H), 0.88(t, 3H), | |
| | | 0.24(s, 9H) | |
| 240 | 125 | | |
| 246 | 128 | 7.29(d, 2H), 7.26(s, 1H), 7.21(s, 1H), 6.58(s, 1H), | |
| | | 6.43(s, 1H), 6.40(d, 1H), 3.29(m, 4H), 2.19-2.01(m, 6H), | |
| | | 1.78(m, 4H), 1.56-1.54(m, 8H), 1.43(s, 2H), | |
| | | 1.33-1.12(m, 18H), 1.02-0.92(m, 20H) | |
| 254 | 268 | | |
| 256 | 200 | 7.29(d, 1H), 7.25(d, 1H), 7.16(d, 1H), 6.59(d, 1H), | 719.47 |
| | | 6.43(d, 1H), 6.40(d, 1H), 3.31(t, 4H), 1.87(m, 8H), | |
| | | 1.76(t, 2H), 1.53(m, 6H), 1.32(s, 6H), 1.21(s, 4H), | |
| | | 1.26-1.20(m, 8H), 0.92(t, 3H), 0.03(s, 9H), −0.04(s, 9H) | |
| 258 | 204 | 7.27(d, 1H), 7.24(s, 2H), 6.58(d, 1H), 6.43(d, 1H), | 675.46 |
| | | 6.426(d, 1H), 4.43(m, 1H), 3.35(t, 2H), 3.09(m, 1H), | |
| | | 1.87(m, 6H), 1.78(m, 3H), 1.56(m, 6H), 1.35(s, 3H), | |
| | | 1.31(s, 3H), 1.24(s, 3H), 1.21(s, 3H), 0.913(t, 3H), | |
| | | 0.94-0.89(m, 2H), 0.62(m, 1H), 0.41(m, 2H), 0.011(s, 9H) | |
| 260 | 238 | 7.27(s, 1H), 7.22(d, 1H), 7.23(s, 1H), 6.57(s, 1H), | 683.43 |
| | | 6.41(d, 1H), 5.47(d, 1H), 3.37-3.29(m, 3H), 2.08(m, 1H), | |
| | | 1.87-1.84(m, 6H), 1.79-1.62(m, 5H), 1.54(m, 6H), | |
| | | 1.53-1.26(m, 4H), 1.35(s, 6H), 1.27(s, 3H), | |
| | | 1.26-1.22(m, 8H), 1.17-1.03(m, 3H), 0.90(t, 3H), | |
| | | 0.66-0.61(m, 1H) | |
| 270 | 230 | | |
| 294 | 300 | | |
| 296 | 278 | | |
| 300 | 180 | | |
| 302 | 198 | | |
| 304 | 120 | | |
| 306 | 250 | | |
| 308 | 208 | | |
| 310 | 204 | | |
| 312 | 210 | | |
| 314 | 218 | | |
| 320 | 178 | | |

### Example 7

### Preparation and Evaluation of Organic EL Devices (Method 1)

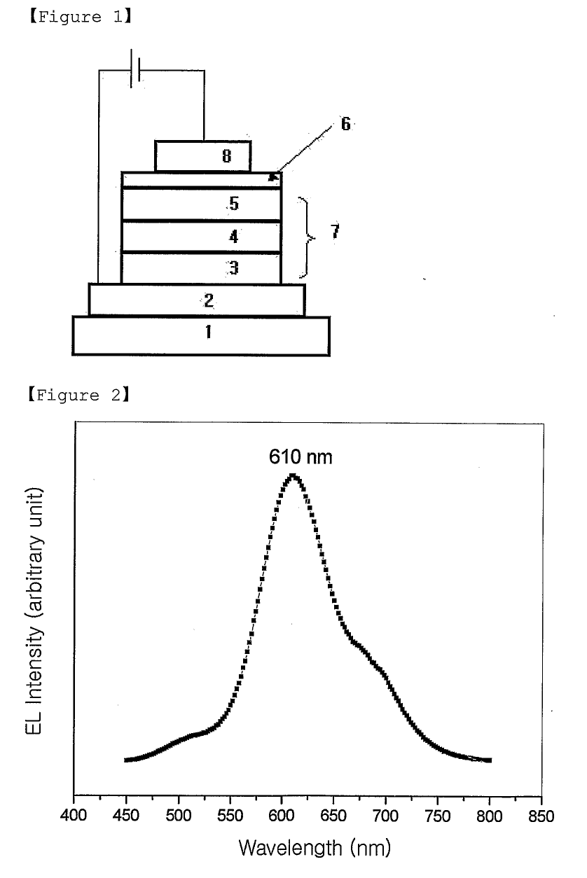

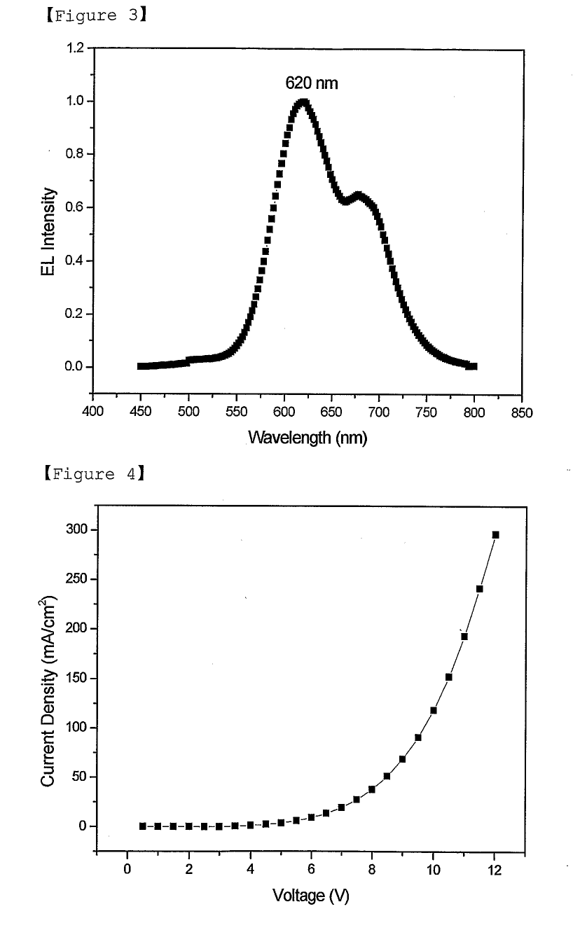

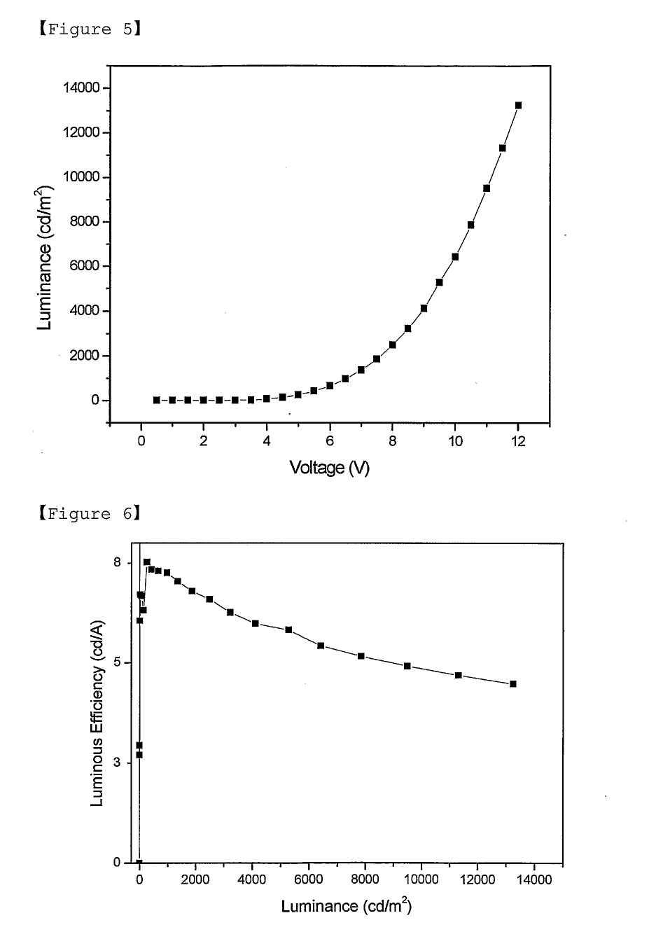

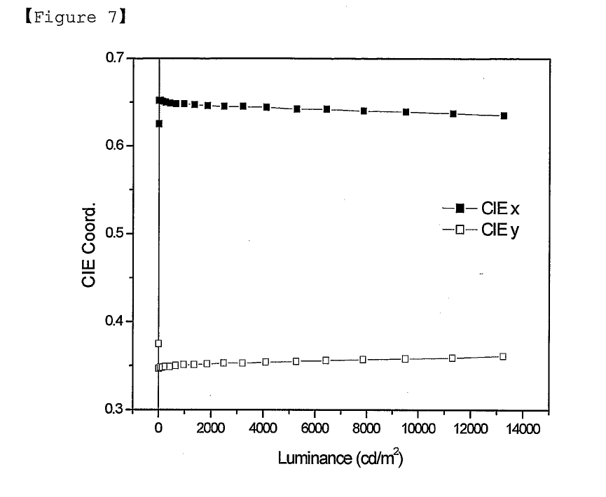

Organic EL devices as shown in FIG. 1 are prepared by using the red electroluminescent compounds synthesized according to the present invention as electroluminescent dopants.

Transparent electrode ITO thin layer (2) (15Ω/□) obtained from the glass (1) (of Samsung-Corning) for organic EL is ultrasonically washed by using trichloroethylene, acetone, ethanol, and distilled water in order, put into isopropanol, kept, and used.

ITO substrate is installed at the substrate folder of a vacuum evaporation equipment, and N,N′-bis(α-naphthyl)-N,N′-diphenyl-4,4′-diamine (NPB) shown in Chemical Formula 106 is put into the cell in the vacuum evaporation equipment, which is then ventilated until the degree of vacuum in the chamber reaches 10⁻⁶ torr. 40-nm-thick hole delivery layer (3) is deposited on the ITO substrate by applying electric current to the cell and evaporating NPB.

Next, tris(8-hydroxyquinoline)-aluminum (Alq) shown in Chemical Formula 107 is put into another cell of the above vacuum evaporation equipment, and electroluminescent dopants synthesized in Examples 1 through 6 are put into another cell. Then, 20-nm-thick electroluminescent layer (4) is deposited on the above hole delivery layer through evaporation and doping of the above two materials at different speeds, where the doping concentration of electroluminescent dopant is 1 to 10 mole % based on that of Alq.

Thereafter, 40-nm-thick Alq is deposited on the above electroluminescent layer as an electron transportation layer (5) in the same method as that of NPB. Further, 2-nm-thick lithium quinolate (Liq) shown in Chemical Formula 110 is deposited further as an electron injection layer (6).

As described in the above, an organic EL device shown in FIG. 1 is prepared by depositing A1 cathode (8) to have a thickness of 150 nm by using another vacuum evaporation equipment after organic layers (7) are formed.

### Example 8

### Preparation and Evaluation of Organic EL Devices (Method 2)

Organic EL devices are prepared by using the red electroluminescent compounds synthesized according to the present invention as electroluminescent dopants.

Transparent electrode ITO thin layer (2) (15Ω/□) obtained from the glass (1) (of Samsung-Corning) for organic EL is ultrasonically washed by using trichloroethylene, acetone, ethanol, and distilled water in order, put into isopropanol, kept, and used.

ITO substrate is installed at the substrate folder of a vacuum evaporation equipment, and N,N′-bis(α-naphthyl)-N,N′-diphenyl-4,4′-diamine (NPB) shown in Chemical Formula 106 is put into the cell in the vacuum evaporation equipment, which is then ventilated until the degree of vacuum in the chamber reaches 10⁻⁶ torr. 40-nm-thick hole delivery layer (3) is deposited on the ITO substrate by applying electric current to the cell and evaporating NPB.

Next, Alq shown in Chemical Formula 107 and rubrene shown in Chemical Formula 108 are put into two other cells in the above vacuum evaporation equipment, and electroluminescent dopants synthesized in Examples 1 through 6 are put into still another cell. Then, 20-nm-thick electroluminescent layer (4) is deposited on the above hole delivery layer through evaporation and doping of the above three materials at different speeds, where the doping concentration of rubrene is 50 to 150 mole %, and that of electroluminescent dopant is 1 to 10 mole % based on that of Alq.

Thereafter, 40-nm-thick Alq is deposited on the above electroluminescent layer as an electron transportation layer (5) in the same method as that of NPB. Further, 2-nm-thick lithium quinolate (Liq) shown in Chemical Formula 110 is deposited further as an electron injection layer (6).

As described in the above, an organic electroluminescent device shown in FIG. 1 is manufactured by depositing A1 cathode (8) to have a thickness of 150 nm by using another vacuum evaporation equipment after organic layers (7) are formed.

### Example 9

### Preparation and Evaluation of Organic EL Devices (Method 3)

Organic EL devices are prepared by using the red electroluminescent compounds manufactured according to the present invention as electroluminescent dopants.

Transparent electrode ITO thin layer (2) (15Ω/□) obtained from the glass (1) (of Samsung-Corning) for organic EL is ultrasonically washed by using trichloroethylene, acetone, ethanol, and distilled water in order, put into isopropanol, kept, and used.

ITO substrate is installed at the substrate folder of a vacuum evaporation equipment, and N,N′-bis(α-naphthyl)-N,N′-diphenyl-4,4′-diamine (NPB) shown in Chemical Formula 106 is put into the cell in the vacuum evaporation equipment, which is then ventilated until the degree of vacuum in the chamber reaches 10⁻⁶ torr. 40-nm-thick hole delivery layer (3) is deposited on the ITO substrate by applying electric current to the cell and evaporating NPB.

Next, Alq shown in Chemical Formula 107 and rubrene shown in Chemical Formula 108 are put into two other cells in the above vacuum evaporation equipment, and electroluminescent dopants synthesized in Examples 1 through 6 are put into still another cell. Then, 20-nm-thick electroluminescent layer (4) is deposited on the above hole delivery layer through evaporation and doping of the above three materials at different speeds, where the doping concentration of rubrene is 50 to 150 mole %, and that of electroluminescent dopant is 1 to 10 mole % based on that of Alq.

Thereafter, 10-nm-thick 2,9-dimethyl-4,7-diphenyl-phenanthroline (BCP) shown in Chemical Formula 109 is deposited on the organic layer as a hole delivery layer. Further, in the same method as that of NPB, 40-nm-thick Alq is deposited on the above electroluminescent layer as an electron transportation layer (5). Still further, 2-nm-thick lithium quinolate (Liq) shown in Chemical Formula 110 is deposited as an electron injection layer (6).

As described in the above, an organic electroluminescent device shown in FIG. 1 is manufactured by depositing A1 cathode (8) to have a thickness of 150 nm by using another vacuum evaporation equipment after organic layers (7) are formed.

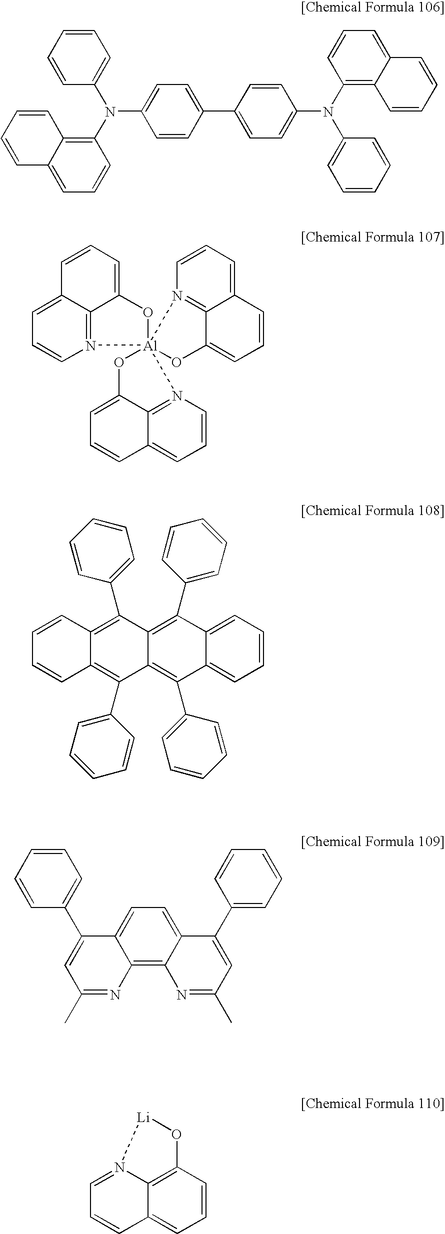

The results of analysis of light-emitting properties of red electroluminescent compounds according to the present invention are presented in Table 6.

Compared to DCJTB which has been known to be a material having the best light-emitting properties, the materials of the present invention have shown significantly improved light-emitting properties. In case of the maximum light-emitting wavelength, they have shown similar wavelength bands generally, and a large number of materials has shown light-emitting peaks at longer wavelength bands compared to DCJTB. It was also confirmed that there was none of peaks of Alq (shown in Chemical Formula 107), which was the host.

Compound groups having silyl substitution radicals showed that the current density of devices was increased, from which it was confirmed that the luminous efficiency was increased as a result. Also, compound groups containing fused rings showed that the luminous efficiency was improved with the light-emitting wavelength maintained almost due to the steric hindrance effect.

In case of compound groups in which the electron acceptor moiety was substituted with an indandion radical instead of dicyano radical, color coordinates were shown to be improved remarkably, where lowering of the luminous efficiency was not accompanied with.

TABLE 6
Results of evaluation of organic EL devices of the
materials developed in the present invention

| | | Efficiency | Current density, | | |
|---|---|---|---|---|---|
| | EL | (cd/A) at | luminance, color | | |
| Comp. | (nm) | 1,000 cd/m² | coordinate at 12 V | | Structure |
| 218 | 610 | 1.34 | 5.7, 70, | (0.611, 0.384) | Alq:Red218 2% |
| | | | | | 400/200/400 |
| 220 | 596 | 5.32 | 11.8, 627, | (0.533, 0.449) | Alq:Red220 1% |
| | | | | | 400/200/400 |
| | 612 | 3.82 | 10.7, 352, | (0.606, 0.392) | Alq70% + Rub 30%:Red220 1% |
| | | | | | 400/200/400 |
| 222 | 604 | 3.21 | 18.1, 587, | (0.554, 0.431) | Alq:Red222 1% |
| | | | | | 400/200/400 |
| 228 | 612 | 1.71 | 38.8, 561, | (0.582, 0.404) | Alq:Red228 1% |
| | | | | | 400/200/400 |
| 230 | 608 | 4.62 | 14.3, 626, | (0.568, 0.420) | Alq:Red230 1% |
| | | | | | 400/200/400 |
| | 618 | 4.0 | 252.5, 7218, | (0.633, 0.364) | Alq + Rub 40%:Red230 2% |
| | | | | | 600/200/300/400 |
| 234 | 604 | 5.5 | 15.4, 741, | (0.57, 0.421) | Alq:Red234 1% |
| | | | | | 400/200/400 |
| | 602 | 4.7 | 269, 11540, | (0.555, 0.42) | Alq:Red234 0.6% |
| | | | | | 400/200/400 |
| | 618 | 1.8 | 208, 3525, | (0.608, 0.381) | Alq:Red234 2% |
| | | | | | 400/200/400 |
| | 612 | 3.65 | 342, 7324, | (0.59, 0.401) | Alq + Rub 30%:Red234 0.6% |
| | | | | | 400/200/400 |
| | 626 | 1.33 | 139.6, 1825, | (0.611, 0.382) | Alq:Red234 3% |
| | | | | | 500/300/500 |
| 236 | 598 | 6.15 | 15.8, 918, | (0.546, 0.443) | Alq:Red236 1% |
| | | | | | 400/200/400 |
| | 608 | 4.2 | 18.1, 682, | (0.578, 0.420) | Alq70% + Rub 30%:Red236 1% |
| | | | | | 400/200/400 |
| 234T | 626 | 4.47 | 266.5, 8765, | (0.631, 0.365) | Alq + Rub 50%:Red234T 2% |
| | | | | | 100/400/300/500 |
| 238 | 612 | 2.28 | 237.4, 4849, | (0.582, 0.408) | Alq:Red238 2% |
| | | | | | 600/200/300/400 |
| | 614 | 2.26 | 161.5, 3577, | (0.593, 0.401) | Alq:Red238 2%/BCP |
| | | | | | 600/200/300/100/300 |
| | 618 | 4.16 | 342.7, 8895, | (0.607, 0.387) | Alq + Rub 40%:Red238 2% |
| | | | | | 600/200/300/400 |
| 250 | 606 | 2.45 | 352, 13510, | (0.553, 0.417) | Alq:Red250 0.6% |
| | | | | | 400/200/400 |
| 254 | 604 | 5.7 | 150, 7409, | (0.537, 0.446) | Alq:Red254 0.6% |
| | | | | | 400/200/400 |
| | 604 | 1.46 | 386.8, 5029, | (0.6, 0.391) | Alq:Red254 2% |
| | | | | | 400/200/400 |
| | 614 | 3.3 | 465.7, 7170, | (0.589, 0.404) | Alq + Rub 30%:Red254 0.6% |
| | | | | | 400/200/400 |
| 256 | 612 | 2.15 | 170, 3474, | (0.599, 0.388) | Alq:Red256 2% |
| | | | | | 400/200/400 |
| | 620 | 4.76 | 272, 8716, | (0.623, 0.372) | Alq + Rub 30%:Red256 2% |
| | | | | | 500/300/500 |
| 260 | 604 | 5.5 | 329, 14910, | (0.548, 0.424) | Alq:Red260 0.6% |
| | | | | | 400/200/400 |
| | 612 | 1.90 | 302.4, 5476, | (0.585, 0.402) | Alq:Red260 2% |
| | | | | | 400/200/400 |
| | 620 | 3.9 | 313, 7520, | (0.61, 0.385) | Alq + Rub 40%:Red260 2% |
| | | | | | 600/200/300/400 |
| 334 | 636 | 4.7 | 317, 11400, | (0.653, 0.340) | Alq + Rubrene 40%:Red 334 2% |
| | | | | | 600/200/300/400 |
| 354 | 638 | 3.6 | 330, 10560, | (0.656, 0.335) | Alq + Rubrene 40%:Red 354 2% |
| | | | | | 600/200/300/400 |
| 335 | 642 | 3.3 | 297, 9520, | (0.660, 0.328) | Alq + Rubrene 40%:Red 335 2% |
| | | | | | 600/200/300/400 |

### INDUSTRIAL APPLICABILITY

As reviewed in detail in the above, compared to the conventional dicyanojulilodyl (DCJ)-group fluorescent materials, red electroluminescent compounds according to the present invention have very superior light-emitting properties, are highly applicable to the manufacture of purely red organic EL panels owing to their superior coloring purity, and are very effective for the manufacture of high-efficiency organic EL panels.

It will be apparent to those skilled in the art that various modifications and variations can be made in the disclosed process and product without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. Organic electroluminescent compounds shown in terms of the following Chemical Formula 1: 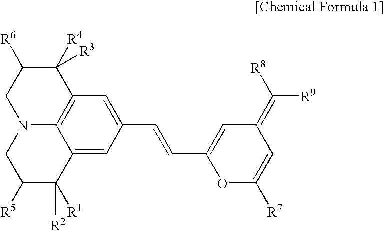
where R¹, R², R³, R⁴, R⁵, and R⁶ are independent from each other, and each of said R¹, R², R³, R⁴, R⁵, and R⁶ may be hydrogen, a side-chained or straight-chained alkyl radical having a chain length of C₁ to C₁₀, cycloalkyl radical of C₅ to C₇, allyl radical, aralkyl radical, fused ring, or R¹¹R¹²R¹³Si—, where
said R¹ and R² or R³ and R⁴ are connected to C₅ to C₁₀ alkylene thus forming a spiro ring;
said R¹ and R⁵ or R³ and R⁶ may form a fused ring as they are connected to C₃ to C₅ alkylene, and carbons of said alkylene of said fused ring connected to said alkylene are substituted with R¹⁴R¹⁵Si<and may form a fused silacycloalkyl radical;
said alkyl radical, cycloalkyl radical, allyl radical, and aralkyl radical of said R¹, R², R³, R⁴, R⁵, and R⁶ may be additionally substituted with more than one R¹¹R¹²R¹³Si—;
R⁷ is hydrogen or a side-chained or straight-chained alkyl radical having a chain length of C₁ to C₁₀, cycloalkyl radical of C₅ to C₇, allyl radical, aralkyl radical, or fused ring;
both of R⁸ and R⁹ is —CN or forms a 1,3-indandion ring as they are combined with 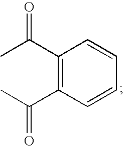
R¹¹, R¹², R¹³, R¹⁴ and R¹⁵ are the same as or different from each other, and each of said R¹¹, R¹², R¹³, R¹⁴ and R¹⁵ may be a side-chained or straight-chained alkyl radical having a chain length of C₁ to C₁₀, cycloalkyl radical of C₅ to C₇, allyl radical, or arallyl radical, where said R¹¹ and R¹² or R¹⁴ and R¹⁵ are connected to alkylene or alkenylene of C₄ to C₁₀ thus forming a spiro ring; and
when more than one of said R¹, R², R³, and R⁴ include said substituent, the remaining substituents among said R¹, R², R³, and R⁴ may be hydrogen, or side-chained or straight-chained alkyl radicals having a chain length of C₁ to C₁₀ that may be substituted or non-substituted, provided that a radical, selected from side-chained or straight-chained alkyl radicals of C₁ to C₁₀ where all of four R¹, R², R³, and R⁴ substitution radicals are composed of hydrogens or of carbons and hydrogens only, is excluded.

2. The organic electroluminescent compounds of claim 1 shown in terms of the following Chemical Formula 2: 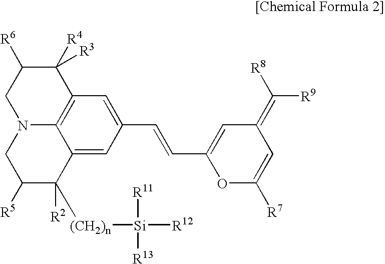
where n is an integer between 0 to 10;
R², R³, R⁴, R⁵, and R⁶ are independent from each other and each of said R², R³, R⁴, R⁵, and R⁶ may be hydrogen, a side-chained or straight-chained alkyl radical having a chain length of C₁ to C₁₀, cycloalkyl radical of C₅ to C₇, allyl radical, aralkyl radical, fused ring, or R¹¹R¹²R¹³Si—, where R³ and R⁴ are connected to C₅ to C₁₀ alkylene thus forming a spiro ring;
said R³ and R⁶ may form a fused ring as they are connected to C₃ to C₅ alkylene, and carbons of the alkylene of the fused ring connected to the above alkylene are substituted with R⁴R⁵Si<and may form a fused silacycloalkyl radical; and
said alkyl radical, cycloalkyl radical, allyl radical, arallyl radical, and fused ring may be additionally substituted with R¹¹R¹²R¹³Si—.

3. The organic electroluminescent compounds of claim 2, wherein:
said R², R³, R⁴, R⁵, and R⁶ are mutually independent hydrogens, methyl, ethyl, n-propyl, i-propyl, n-butyl, i-butyl, t-butyl, n-pentyl, i-amyl, n-hexyl, n-heptyl, n-octyl, 2-ethylhexyl, n-nonyl, cycloalkyl radicals such as cyclopentyl, 2-methylcyclopentyl, 3-methylcyclohexyl, cycloheptyl, etc., phenyl, toluoyl, naphthyl, benzyl, 3-phenylpropyl, 2-phenylpropyl, adamantyl, 4-pentylbicyclo[2,2,2]octyl, norbornene, trimethylsilyl, triethylsilyl, tri(n-propyl)silyl, tri(i-propyl)silyl, tri(n-butyl)silyl, tri(i-butyl)silyl, tri(t-butyl)silyl, tri(n-pentyl)silyl, tri(i-amyl)silyl, t-butyldimethylsilyl, triphenylsilyl, tri(p-tolu)silyl, and dimethylcyclohexylsilyl;
where R¹¹ and R¹² may form methylsilacyclopentyl, methylsilacyclopentenyl, methylsilacyclohexyl, or ethylsilacyclohexyl radical connected to alkylene or alkenylene; and
said alkyl radical, cycloalkyl radical, allyl radical, aralkyl radical, and fused ring may be additionally substituted with trimethylsilyl, triethylsilyl, tri(n-propyl)silyl, tri(i-propyl)silyl, tri(n-butyl)silyl, tri(i-butyl)silyl, tri(t-butyl)silyl, tri(n-pentyl)silyl, tri(i-amyl)silyl, t-butyldimethylsilyl, triphenylsilyl, tri(p-tolu)silyl, or dimethylcyclohexylsilyl

4. The organic electroluminescent compounds of claim 1 shown in terms of the following Chemical Formula 3 or 4: 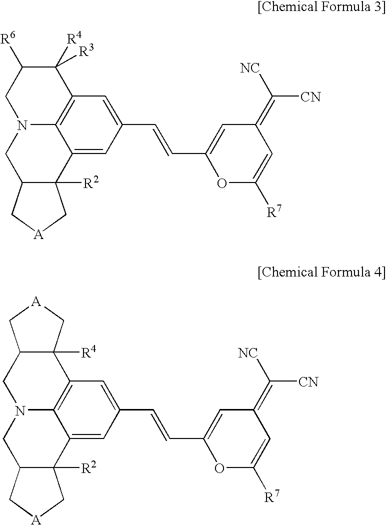
where substituents R², R³, R⁴, R⁶, and R⁷ are the same as those in Chemical Formula 1 or 2; said A may be mutually independent —CH₂—, —CH₂CH₂—, —CH₂CH₂CH₂—, etc.; and one of carbons in alkylene of the fused ring including A may be substituted with R¹⁴R¹⁵Si<forming a fused silacycloalkyl radical.

5. The organic electroluminescent compounds of claim 4 shown in terms of the following Chemical Formula 5 or 6: 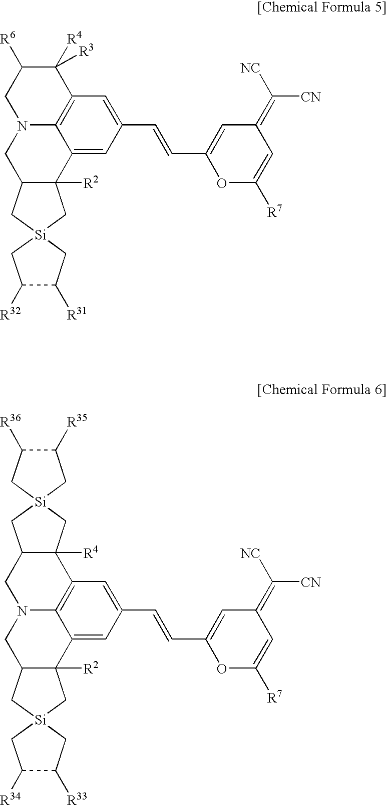
where “---” refers to a single bond or a double bond; R³¹, R³², R³³, R³⁴, R³⁵ and R³⁶ are independent from each other and are hydrogen, straight-chained or side-chained alkyl radical of C₁ to C₅; and R², R³, R⁴, R⁶, and R⁷ are the same as the substituents of Chemical Formula 1 or 2.

6. The organic electroluminescent compounds of claim 1 including a spiro ring shown in terms of the following Chemical Formula 7 or 8: 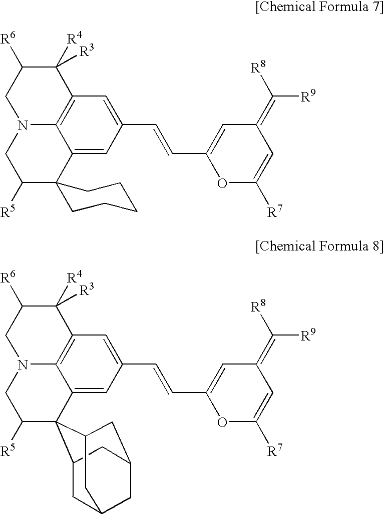
where R³, R⁴, R⁵, R⁶, R⁷, R⁸ and R⁹ are the same as those shown in Chemical Formula 2.

7. The organic electroluminescent compounds of claim 1 shown in terms of the following Chemical Formula 9:

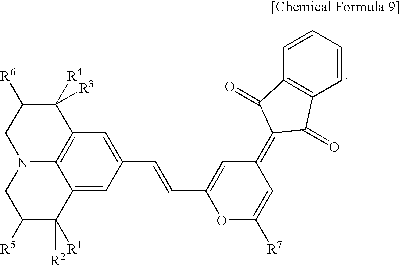

8. The organic electroluminescent compounds of claim 1, wherein:
said R⁷ is independent from each other and is selected from hydrogen, methyl, ethyl, n-propyl, i-propyl, n-butyl, i-butyl, t-butyl, n-pentyl, i-amyl, n-hexyl, n-heptyl, n-octyl, 2-ethylhexyl, n-nonyl, cyclopentyl, 2-methylcyclopentyl, 3-methylcyclohexyl, cycloheptyl, phenyl, toluoyl, naphthyl, benzyl, 3-phenylpropyl, 2-phenylpropyl, adamantyl, 4-pentyl bicyclo[2,2,2]octyl, and norbornene.

9. The organic electroluminescent compounds of claim 1 selected from the following compounds:

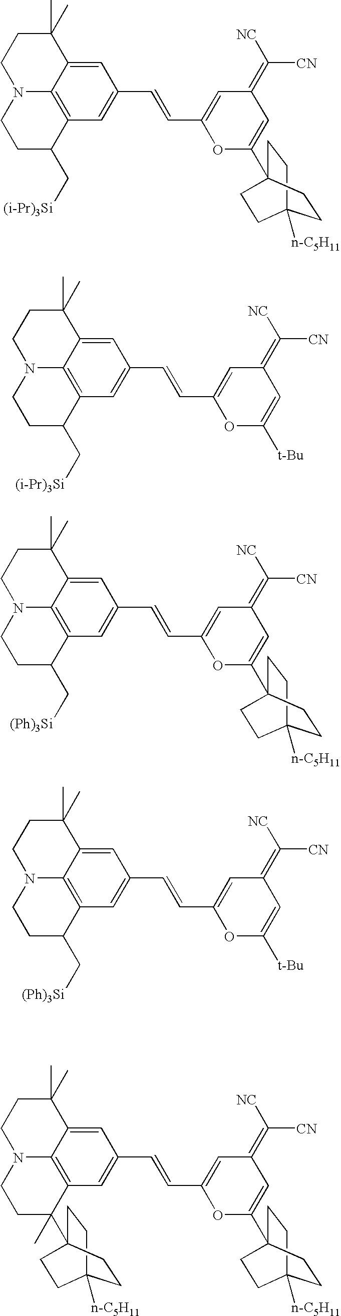

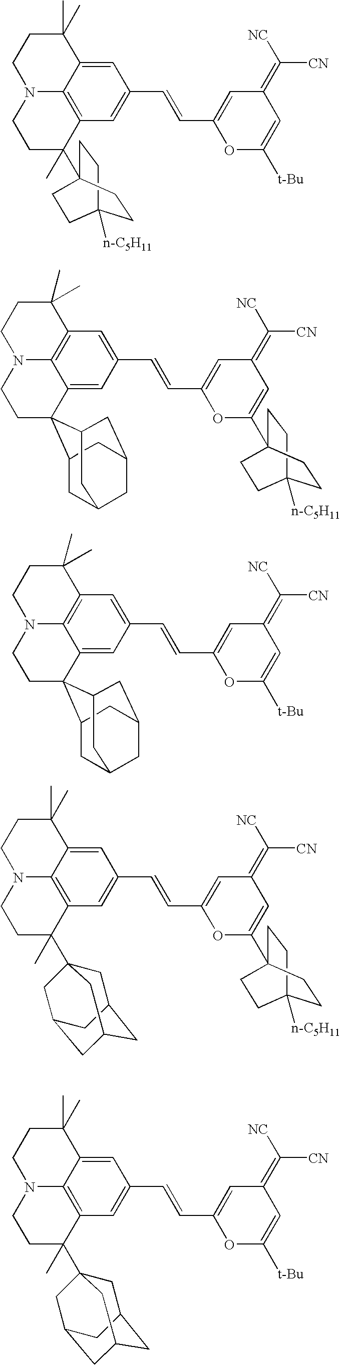

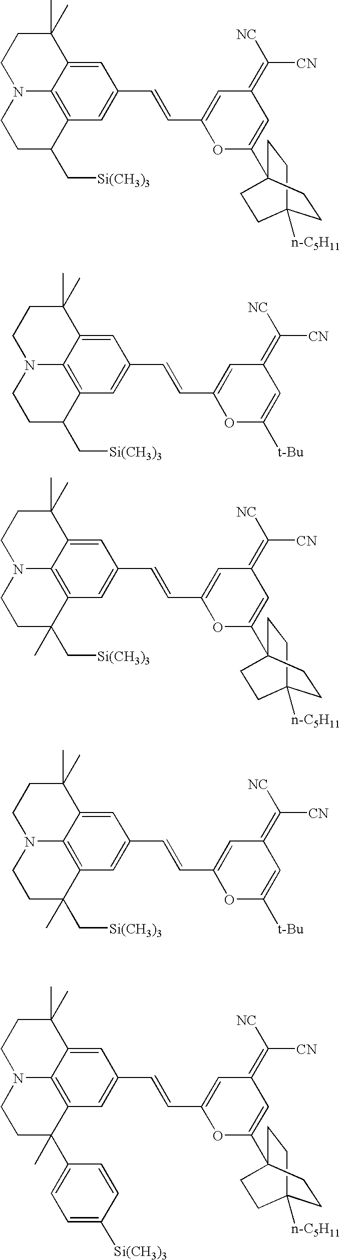

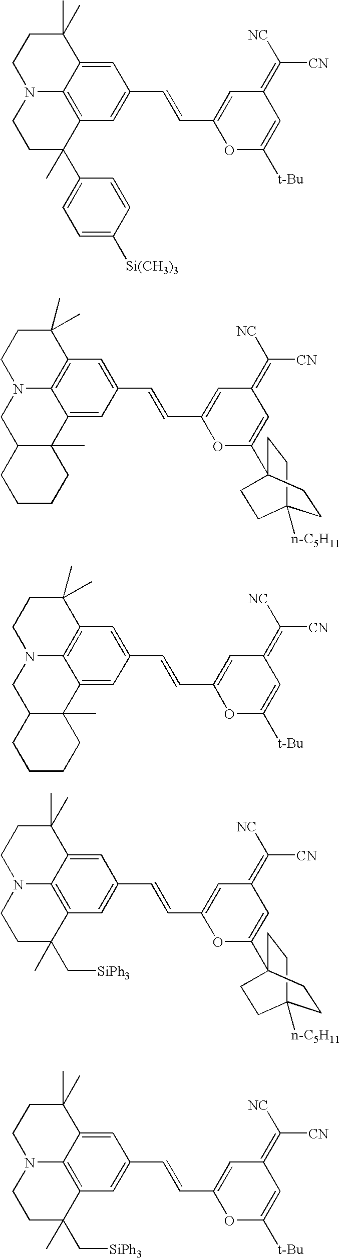

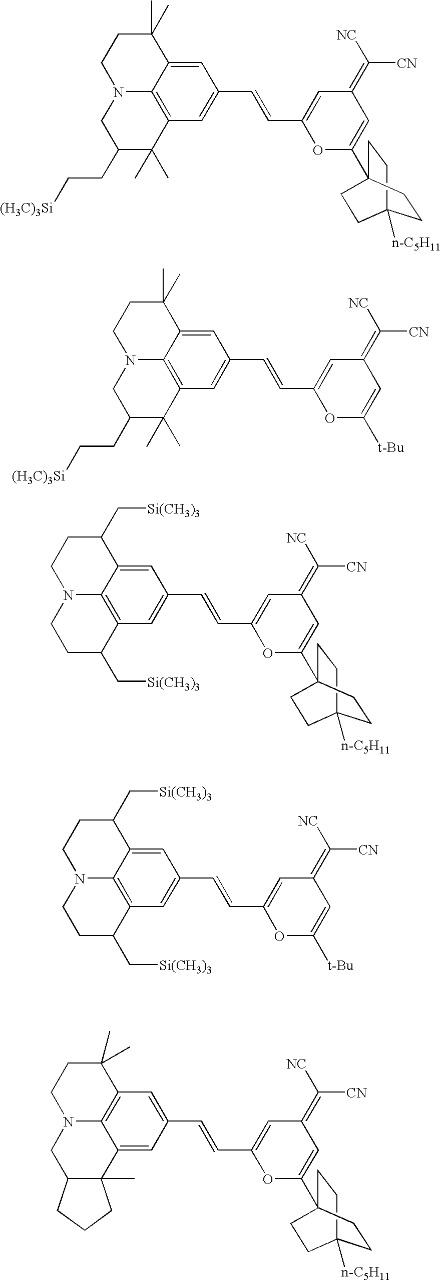

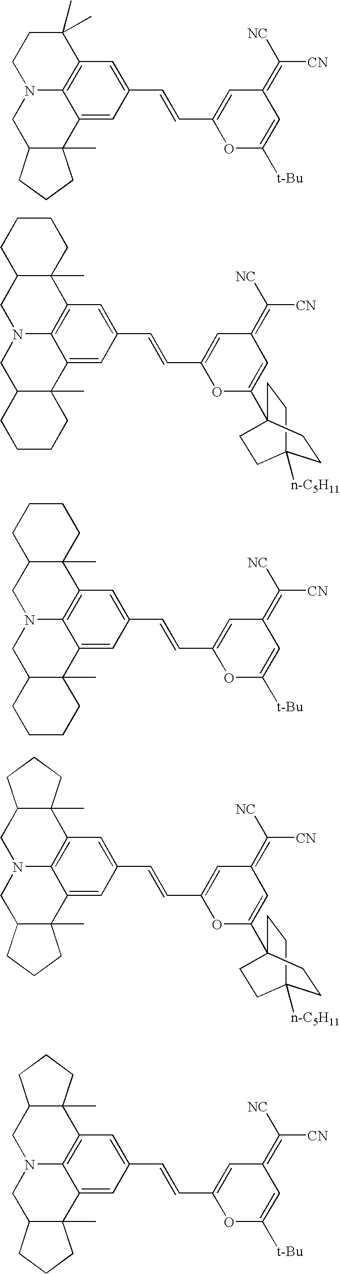

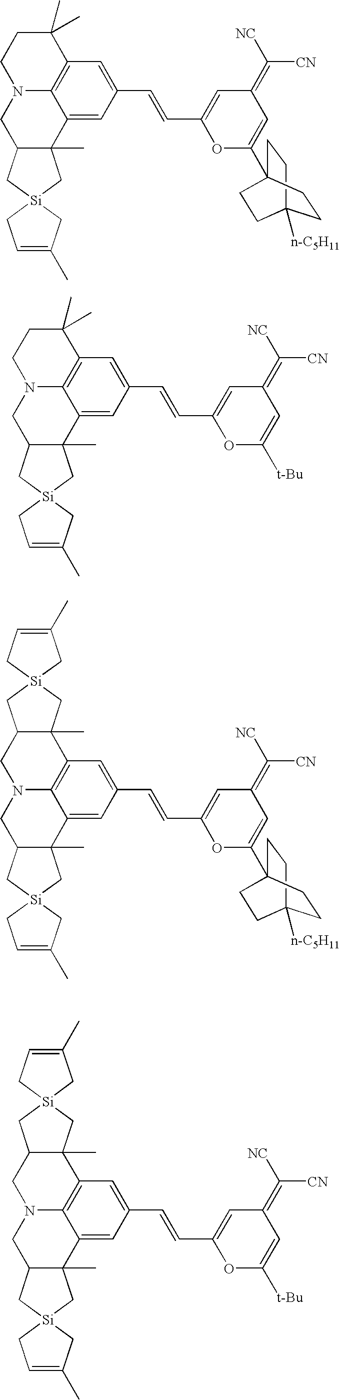

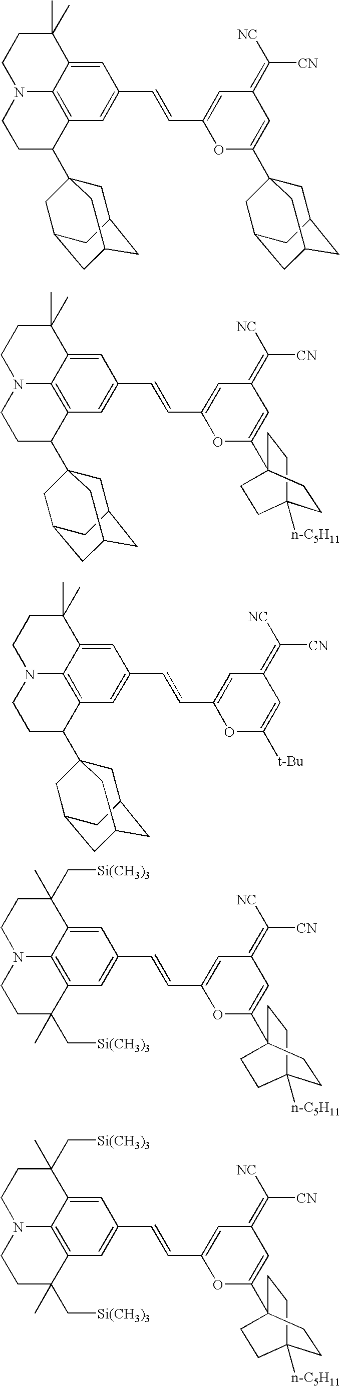

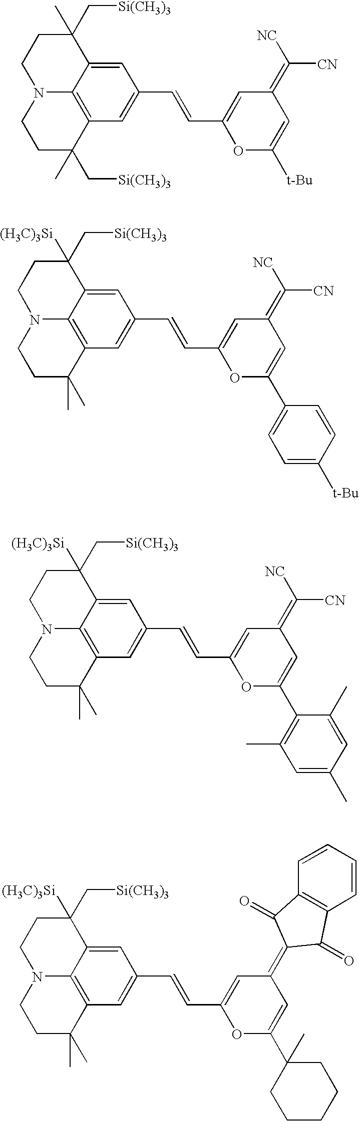

10. The method of preparation of said organic electroluminescent compounds shown in said Chemical Formula 1 of claim 1, comprising the steps shown in Chemical Equation 11 as follows:
a) synthesis of a tetrahydroquinoline derivative (4) by reacting an aniline derivative with benzotriazolyl methane substituted (2) and an alkene derivative (3);
b) synthesis of a julilodyl derivative (7) by making a tetrahydroquinoline derivative with benzotriazolyl methane substituted (5) from said tetrahydroquinoline derivative (4) and reacting said tetrahydroquinoline derivative with benzotriazolyl methane substituted (5) with another alkene derivative (6);
c) synthesis of a julilodyl aldehyde derivative (8) from said julilodyl derivative (7); and
d) reacting said julilodyl aldehyde derivative (8) with a pyran derivative.

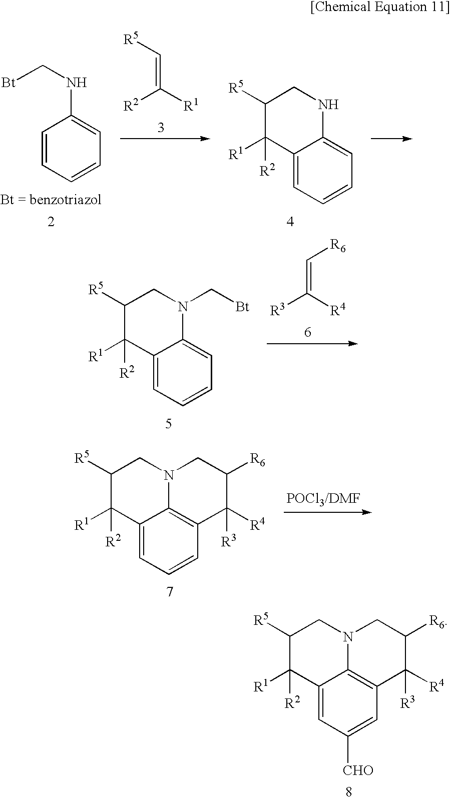

11. The method of preparation of said organic electroluminescent compounds shown in Chemical Formula 1 of claim 1, comprising the step of manufacture of a julilodyl derivative (9) by reacting di(benzotriazolylmethyl)phenyl amine (2) and said alkene derivative (3) as shown in Chemical Equation 12:

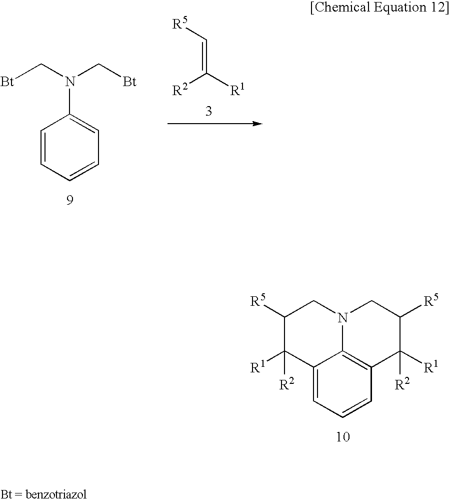

12. Organic electroluminescent devices characterized by including said organic electroluminescent compounds of any of claims 1 through 9 as red electroluminescent materials.
